(19) United States
(12) Patent Application Publication
(10) Pub. No.: US 2012/0045515 A1
(43) Pub. Date: Feb. 23, 2012

(54) HOLLOW SILICA PARTICLE WITH A POLYMER THEREON

(76) Inventors: Ye Liu, Singapore (SG); Decheng Wu, Singapore (SG)

(21) Appl. No.: 13/148,046

(22) PCT Filed: Jan. 25, 2010
(86) PCT No.: PCT/SG10/00021

§ 371 Date: Oct. 24, 2011

Related U.S. Application Data
(60) Provisional application No. 61/149,777, filed on Feb. 4, 2009.

(51) Int. Cl. A61K 9/14; A61P 35/00; C07F 7/10; B82Y 5/00

(52) U.S. Cl. 424/489; 556/411; 977/773

(57) ABSTRACT

The present invention provides a hollow silica micro- or nanoparticle with a polymer immobilized thereon. The polymer is covalently linked to the silica particle via urethane groups. Provided is also a method of covalently coupling a polymer to a silica surface. The method comprises contacting a silica surface that carries amino functional groups with a polymer with a carbonate group of the general Formula (2). R² in Formula (2) is the polymer and R³ is one of an aliphatic, an alicyclic an aromatic and an arylaliphatic group or a silyl group with a main chain of about 1 to about 20 carbon atoms and 0 to about 6 heteroatoms selected from the group consisting of N, O, S, Se and Si. The method further comprises allowing the carbonate group of the polymer and an amino functional group on the silica surface to undergo a coupling reaction, thereby covalently coupling the polymer to the silica surface.

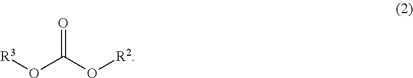

### CROSS REFERENCE TO RELATED APPLICATIONS

This application makes reference to and claims the benefit of priority of an application for “Polymers Functionalized Hollow Silica and the Methods for Polymers Functionalized Surfaces” filed on Feb. 4, 2009 with the United States Patent and Trademark Office, and there duly assigned serial number U.S. Provisional 61/149,777. The content of said application filed on Feb. 4, 2009 is incorporated herein by reference for all purposes, including an incorporation of any element or part of the description, claims or drawings not contained herein and referred to in Rule 20.5(a) of the PCT, pursuant to Rule 4.18 of the PCT.

### FIELD OF THE INVENTION

The present invention relates to a hollow silica particle with a polymer thereon. The polymer is linked to the hollow silica particle. The invention further provides a method that is suitable for the formation of such a hollow silica particle.

### BACKGROUND OF THE INVENTION

In recent years, there has been much interest in the fabrication of hollow silica spheres. These hollow spheres often exhibit properties that are substantially different from those of general particles (for example, their low density, large specific surface area, stability, and surface permeability), thus making them attractive from both a scientific and a technological viewpoint. Applications for such spheres are in cosmetics, as capsule agents for drug delivery systems (DDS), in catalysis, coatings, composite materials, dyes, inks, artificial cells, fillers, and protection for sensitive agents such as enzymes and proteins.

There are a variety of methods for fabricating a wide range of stable, hollow spheres of various compositions. Various groups have produced hollow spheres using emulsion polymerization or inter-facial polymerization strategies. Hollow monodisperse cross-linked polymer particles were produced by suspension polymerization of emulsion droplets with polystyrene dissolved in an aqueous solution of poly(vinyl alcohol). Polymer hollow spheres were obtained by cross-linking polymerization of hydrophobic monomers in the interior of the surfactant bilayer of vesicles (Journal of Colloid and Interface Science (2003) 266, 107-114).

The application and commercialization of hollow spherical structures have, however, been limited, mainly because of the disadvantages associated with the techniques for their production. Relatively complex experimental procedures are employed in some preparation methods. There remains therefore a need for a method that allows easy preparation of hollow structures so that it can be used in various industrial fields.

Nanosized hollow silica spheres can be used as fillers to prepare porous materials having low weight, low dielectric constant, and heating insulation capacity. Also hollow silica can be applied as carriers of active species for encapsulation and controlled delivery. However hollow silica materials have fatal shortcomings. A tendency of silica to easily aggregate and poor dispersion in solvents hinder the application of such silica. In addition, poor processability of hollow silica results in heterogeneous products.

On the other hand, organic vesicles formed via self-assembly of lipids or amphiphilic copolymers are expected to be mimics of nature biological vesicles, and are mainly applied for encapsulation and delivery of active species. Liposomes are vesicles that have concentrically ordered lipid bilayers which encapsulate an aqueous phase and can be used for drug encapsulation into liposomes, but lipid stability is important in the development of liposomal drug delivery systems and the stability of liposomes from lipids is poor. Polymer vesicles are microscopic sacs that enclose a volume with a molecularly thin membrane. The membranes are generally self-directed assemblies of amphiphilic molecules with dual hydrophilic-hydrophobic character. Structural features of polymer vesicles, as well as their properties, including stability, fluidity, and intermembrane dynamics, are greatly influenced by characteristics of the polymers. Polymer vesicles without sufficiently thick hydrophobic segments are unstable and when the hydrophobic segments are thick enough to provide stable polymer vesicles, the fluidity of the vesicles become very low so the encapsulated species can not be released. Hence, so far a dilemma between stability and fluidity of vesicles remains, i.e., vesicles with good fluidity are instable and vice versa.

For encapsulation and delivery of active species, poor dispersion of hollow silica in aqueous solution is a significant hurdle.

Grafting of poly(ethylene glycol) (PEG) to silica is an important approach for surface modification. The grafted PEG can improve the hydrophilic degree of silica and also prevent nonspecific adsorption of proteins to silica surfaces. These functions can improve solubility of silica particles and hollow silica in aqueous solution, and increase the circulation time of silica species in blood. However, both achieving (i) a high density and (ii) stability in water of the grafted polymer on the surface have remained a challenge. As reviewed by Zhang et al. (Journal of Colloid and Interface Science (2007) 310, 446-455) detachment of PEG from the surface has been attributed to inter alia degradation of the respective layer formed on the silica surface, including hydrolysis of urethane groups used as a link. These authors suggested coupling of N-succinimidyl esters carrying mono-methoxy poly(ethylene glycol) to the silica surface, which is, however, a cost intensive approach. Grafting densities were not increased compared to previous methods.

Yang et al. (Langmuir (2008) 24, 3417) reported that PEG was grafted to hollow silica via carboxyl terminated PEG (PEG-diCOOH), obtained by using succinic anhydride. The linkage used was via amide and ester groups, albeit no clear indication of the grafting was given. Other reported methods of grafting PEG to silica include: grafting of PEG to the surface of silica particles via the emulsion method using PEG-IPTES (3-(triethoxysiyl)propyl isocyanate (Materials Letter (2005) 59, 929), wherein the urethane linkage was formed in advance in PEG-IPTES; grafting of PEG via the reaction of the amine with PEG-NHS (Chemistry of Materials (2007) 19, 5074), wherein PEH-NHS is expensive; grafting of PEG to silica via the reaction of N-(Triethoxysilylpropyl)-O-poly-(ethylene oxide)urethane with the Si—OH (Anal. Chem. (2006) 78, 4326).

It is therefore an object of the present invention to provide a hollow particle with properties that overcome at least some of the above described disadvantages. This object is solved by the particle of claim 1.

### SUMMARY OF THE INVENTION

In a first aspect the present invention provides a hollow silica micro- or nanoparticle with a polymer immobilized thereon. The polymer is covalently linked, e.g. grafted, to the silica particle via urethane groups. Generally the polymer is covalently linked to the silica particle via one or more bridges that have an urethane group. The bridge may be taken to include the urethane group, typically at terminal position.

In a second aspect the invention provides a pharmaceutical composition. The pharmaceutical composition includes a plurality of hollow silica particles. Typically the hollow silica particles have an inner void that includes a pharmaceutically active compound.

In a third aspect the invention provides a method of covalently coupling a polymer to a silica surface. The method includes providing a silica surface. The silica surface carries amino functional groups. The method also includes providing a polymer. The polymer has a carbonate group. It can be represented by the general Formula (2):

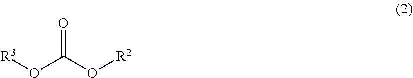

In general Formula (2) R² is the polymer. R³ is an aliphatic, an alicyclic, an aromatic, an arylaliphatic group or a silyl group. This aliphatic, alicyclic, aromatic, arylaliphatic group or silyl group has a main chain of about 1 to about 20 carbon atoms. The main chain may further have 0 to about 6 heteroatoms. The heteroatoms may be selected from N, O, S, Se and Si. The method further includes contacting the polymer and the silica surface. The method also includes allowing the carbonate group of the polymer and an amino functional group on the silica surface to undergo a coupling reaction. By allowing this coupling reaction to occur, the polymer is covalently coupled to the silica surface.

The invention will be better understood with reference to the detailed description when considered in conjunction with the accompanying drawings.

### BRIEF DESCRIPTION OF THE DRAWINGS

FIG. 1 is a scheme on the preparation of PEG-g-hollow silica vesicles according to the invention.

FIG. 2A is a TEM image of pristine hollow silica spheres. The white bar represents 100 nm. FIG. 2B depicts TGA curves of a) pristine hollow silica spheres; b) TMSPEA attached hollow silica spheres; and c) PEG-g-hollow silica vesicles. FIG. 2C is a TEM image of PEG-g-hollow silica vesicles. The white bar represents 100 nm. FIG. 2D shows the dependence of the apparent diffusion coefficient, Dₐₚₚ, of PEG-g-hollow silica vesicles on the square of the wave scattering vector, q², recorded in aqueous solution. The linearly fitting result is presented for guidance.

FIG. 3 shows the release profile of a) paclitaxel loaded in PEG-g-hollow silica vesicles, and b) free paclitaxel in 1×PBS buffer solution at 37° C.

FIGS. 4A-4F show photographs of a confocal fluorescent microscope image of MCF-7 cells after incubation with FITC labelled PEG-g-hollow silica vesicles for 18 h. Cell nuclei were stained with DAPI. FIG. 4A is a representation of the image without fluorescence signals. FIG. 4B depicts the image together with fluorescence signals. FIG. 4C depicts only the green fluorescence signals of the same image. To facilitate relating the signals to the positions in the context of MCF-7 cells FIG. 4D shows the green fluorescence signals against the photograph without fluorescence signals (FIG. 4A). FIG. 4E depicts only the blue fluorescence signals of the same image. FIG. 4F shows the blue fluorescence signals against the photograph without fluorescence signals (FIG. 4A) for a comparison.

FIGS. 4G-4L show a photograph of a confocal fluorescent microscope image of U-87 MG cells after incubation with FITC labelled PEG-g-hollow silica vesicles for 18 h. Cell nuclei were stained with DAPI. FIG. 4G is a representation of the image without fluorescence signals. FIG. 4H depicts the image together with fluorescence signals. FIG. 4I depicts only the green fluorescence signals of the same image. FIG. 4J shows the green fluorescence signals against the photograph without fluorescence signals (FIG. 4G) in order to facilitate identification of the positions of the signals in the context of U-87 MG cells. FIG. 4K depicts only the blue fluorescence signals of the image of U-87 MG cells. FIG. 4L shows the blue fluorescence signals against the photograph without fluorescence signals (FIG. 4G) for a comparison.

FIG. 5A depicts an analysis of the in vitro cytotoxicity of PEG-g-hollow silica vesicles after incubation for 72 h in a) MCF-7 cells and b) U-87 MG cells. FIG. 5B depicts an analysis of the in vitro cytotoxicity of free paclitaxel and paclitaxel loaded PEG-g-hollow silica vesicles after incubation for 72 h in MCF-7 cells and U-87 MG cells.

FIG. 6A depicts nitrogen adsorption-desorption isotherms for hollow silica spheres.

FIG. 6B depicts a pore size distribution for hollow silica measured using the BJH method.

FIG. 7 depicts FTIR spectra of a) hollow silica spheres and b) PEG-g-hollow silica vesicles.

FIG. 8 depicts a ¹H NMR spectrum of PEG-g-hollow silica vesicles recorded in deuterium oxide.

FIG. 9 shows a comparison of IC₅₀ values (μg/ml) of paclitaxel loaded PEG-g-hollow silica vesicles and free paclitaxel in cancer cells after different incubation times.

### DETAILED DESCRIPTION OF THE INVENTION

The present invention is based on the finding that a surprisingly high polymer content on a surface, i.e. a high density of the polymer, can be achieved when a polymer is grafted onto a silica surface via urethane linking moieties. Comparably low cost chemicals have further been identified that can be used in the formation of this linkage. Unexpectedly, the linkage is even particularly stable on silica particles in aqueous solution. When applied onto hollow silica particles there may be provided carriers for various matter such as a marker or a pharmaceutically active compound in vivo, ex vivo and in vitro. By including selected ligands onto the polymer surface or (depending on the size of the ligand) to the silica surface, a respective particle can for instance be used as a probe or a “Trojan horse”-like carrier in the human or animal body in diagnosis and treatment of disease, for example.

It is further known that immobilizing, including covalently grafting, hydrophilic, i.e. generally polar, polymer chains or oligomers with brush-like structures onto surfaces renders these surfaces nonadhesive. When in contact with a biological fluid a respective surface resists unintended accumulation of biological entities, which can result in the formation of a biofilm. On exposure of a synthetic surface to biological media, macromolecular moieties are firstly adsorbed rapidly, thereby forming a conditioning film for the subsequent adhesion of other molecular and cellular entities. For a particle with a polymer immobilized thereon according to the invention this initial and nonspecific adsorption of macromolecular components, notably proteins, biological cells, and microorganisms is prevented. A corresponding particle is therefore well suited for e.g. biosensors and both biomedical and nonbiomedical applications. These and other advantages will become more readily apparent from the following explanations and the appended drawings.

The polymer is linked to the hollow silica particle by means of an urethane moiety. A respective urethane moiety is generally of the following structure:

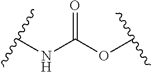

The symbol [symbol] in this formula defines that the respective bond continues, but is not further shown. One or more bridges that have such an urethane group provide a covalent attachment of the polymer to the silica surface. Accordingly, generally such an urethane moiety is included in a bridge via which the polymer is covalently linked to the silica particle. A respective bridge is generally hydrocarbon based, although it may include one or more atoms in its main chain, as well as in any side chain if present, that differ(s) from carbon. In some embodiments the bridge includes a nitrogen atom in its main chain, for instance in the form of a secondary amino group, i.e. the moiety —NH—. The bridge may for example have a single, two or more such secondary amino groups. The bridge may for example be of aliphatic or arylaliphatic nature and include aliphatic, aromatic and alicyclic elements. The bridge may for example have a main chain with a length of 1 to about 30 carbon atoms such as 2 to about 30 carbon atoms or 2 to about 25 carbon atoms, including 1 to about 25 carbon atoms, 1 to about 20 carbon atoms, about 2 to about 20 carbon atoms, about 3 to about 20 carbon atoms, about 1 to about 15 carbon atoms, about 2 to about 15 carbon atoms, about 1 to about 10 carbon atoms, about 2 to about 10 carbon atoms or about 1 to about 10 carbon atoms, such as 1, 2, 3, 4, 5, 6, 7, 8, 9, 10, 11, 12, 13, 14, 15, 16, 17, 18, 19 or 20 carbon atoms. The main chain of the aliphatic, alicyclic, aromatic or arylaliphatic bridge of R¹ may include 0 to about 12 heteroatoms, such as 0 to about 10 heteroatoms, 0 to about 9 heteroatoms, 0 to about 8 heteroatoms, 0 to about 7 or 0 to about 6, e.g. 0 to about 5 or 0 to about 4, including 0, 1, 2, 3, 4, 5, 6, 7, 8 or 9 heteroatoms.

In some embodiments the bridge that couples the polymer to the silica surface has one end that is covalently coupled to the silica surface. The bridge may have a further end defined by an urethane moiety. The urethane moiety is covalently linked to the polymer. The link of the polymer to the hollow silica particle can in some embodiments accordingly be represented by the following formula (1):

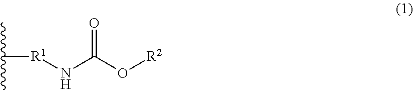

In formula (1) the symbol [symbol] defines that the bond continues, but is not further shown. Typically this covalent bond is a bond to the silica particle. R² is the respective polymer. R¹ is an aliphatic, alicyclic, aromatic or arylaliphatic bridge. The aliphatic, alicyclic, aromatic or arylaliphatic bridge has a main chain of 1 to about 30 carbon atoms such as 2 to about 30 carbon atoms or 2 to about 25 carbon atoms, including about 1 to about 20 carbon atoms, about 2 to about 20 carbon atoms, about 3 to about 20 carbon atoms, about 1 to about 15 carbon atoms, about 2 to about 15 carbon atoms, about 1 to about 10 carbon atoms, about 2 to about 10 carbon atoms or about 1 to about 10 carbon atoms, such as 1, 2, 3, 4, 5, 6, 7, 8, 9, 10, 11, 12, 13, 14, 15, 16, 17, 18, 19 or 20 carbon atoms. The main chain of the aliphatic, alicyclic, aromatic or arylaliphatic bridge of R¹ may include 0 to about 10 heteroatoms, such as 0 to about 8 heteroatoms, 0 to about 7 or 0 to about 6, e.g. 0 to about 5 or 0 to about 4, including 0, 1, 2, 3, 4, 5 or 6 heteroatoms. A heteroatom is any other atom than carbon and hydrogen, such as N, O, S, Se and Si.

The term “aliphatic” means, unless otherwise stated, a straight or branched hydrocarbon chain, which may be saturated or mono- or poly-unsaturated and include heteroatoms. The term “heteroatom” as used herein means an atom of any element other than carbon or hydrogen. An unsaturated aliphatic bridge or group contains one or more double and/or triple bonds (alkenyl or alkinyl moieties). The branches of the hydrocarbon chain may include linear chains as well as non-aromatic cyclic elements. The hydrocarbon chain, which may, unless otherwise stated, be of any length, and contain any number of branches. Typically, the hydrocarbon (main) chain includes 1 to 5, to 10, to 15 or to 20 carbon atoms. Examples of alkenyl radicals are straight-chain or branched hydrocarbon radicals which contain one or more double bonds. Alkenyl radicals generally contain about two to about twenty carbon atoms and one or more, for instance two, double bonds, such as about two to about ten carbon atoms, and one double bond. Alkynyl radicals normally contain about two to about twenty carbon atoms and one or more, for example two, triple bonds, preferably such as two to ten carbon atoms, and one triple bond. Examples of alkynyl radicals are straight-chain or branched hydrocarbon radicals which contain one or more triple bonds. Examples of alkyl groups are methyl, ethyl, propyl, butyl, pentyl, hexyl, heptyl, octyl, nonyl, decyl, the n isomers of these radicals, isopropyl, isobutyl, isopentyl, sec-butyl, tert-butyl, neopentyl, 3,3 dimethylbutyl. Both the main chain as well as the branches may furthermore contain heteroatoms as for instance N, O, S, Se or Si or carbon atoms may be replaced by these heteroatoms.

The term “alicyclic” may also be referred to as “cycloaliphatic” and means, unless stated otherwise, a non-aromatic cyclic moiety (e.g. hydrocarbon moiety), which may be saturated or mono- or poly-unsaturated. The cyclic hydrocarbon moiety may also include fused cyclic ring systems such as decalin and may also be substituted with non-aromatic cyclic as well as chain elements. The main chain of the cyclic hydrocarbon moiety may, unless otherwise stated, be of any length and contain any number of non-aromatic cyclic and chain elements. Typically, the hydrocarbon (main) chain includes 3, 4, 5, 6, 7 or 8 main chain atoms in one cycle. Examples of such moieties include, but are not limited to, cyclopentyl, cyclohexyl, cycloheptyl, or cyclooctyl. Both the cyclic hydrocarbon moiety and, if present, any cyclic and/or linear substituents may furthermore contain heteroatoms, as for instance N, O, S, Se or Si, or a carbon atom may be replaced by these heteroatoms. The term “alicyclic” also includes cycloalkenyl moieties that are unsaturated cyclic hydrocarbons, which generally contain about three to about eight ring carbon atoms, for example five or six ring carbon atoms. Cycloalkenyl radicals typically have a double bond in the respective ring system. Cycloalkenyl radicals may in turn be substituted. Examples of such moieties include, but are not limited to, cyclohexenyl, cyclooctenyl or cyclodecenyl.

In contrast thereto, the term “aromatic” means an at least essentially planar cyclic hydrocarbon moiety of conjugated double bonds, which may be a single ring or include multiple condensed (fused) or covalently linked rings, for example, 2, 3 or 4 fused rings. The term aromatic also includes alkylaryl. Typically, the hydrocarbon (main) chain includes 5, 6, 7 or 8 main chain atoms in one cycle. Examples of such moieties include, but are not limited to, cyclopentadienyl, phenyl, napthalenyl-, [10]annulenyl-(1,3,5,7,9-cyclodecapentaenyl-), [12]annulenyl-, [8]annulenyl-, phenalene (perinaphthene), 1,9-dihydropyrene, chrysene (1,2-benzophenanthrene). An example of an alkylaryl moiety is benzyl. The main chain of the cyclic hydrocarbon moiety may, unless otherwise stated, be of any length and contain any number of heteroatoms, as for instance N, O and S. Such a heteroaromatic moietie may for example be a 5- to 7-membered unsaturated heterocycle which has one or more heteroatoms from the series O, N, S. Examples of such heteroaromatic moieties (which are known to the person skilled in the art) include, but are not limited to, furanyl-, thiophenyl-, naphtyl-, naphthofuranyl-, anthrathiophenyl-, pyridinyl-, pyrrolyl-, quinolinyl, naphthoquinolinyl-, quinoxalinyl-, indolyl-, benzindolyl-, imidazolyl-, oxazolyl-, oxoninyl-, oxepinyl-, benzoxepinyl-, azepinyl-, thiepinyl-, selenepinyl-, thioninyl-, azecinyl-, (azacyclodecapentaenyl-), diazecinyl-, azacyclododeca-1,3,5,7,9,11-hexaene-5,9-diyl-, azozinyl-, diazocinyl-, benzazocinyl-, azecinyl-, azaundecinyl-, thia[11]annulenyl-, oxacyclotrideca-2,4,6,8,10,12-hexaenyl- or triazaanthracenyl-moieties.

By the term “arylaliphatic” is meant a hydrocarbon moiety, in which one or more aromatic moieties are substituted with one or more aliphatic groups. Thus the term “arylaliphatic” also includes hydrocarbon moieties, in which two or more aryl groups are connected via one or more aliphatic chain or chains of any length, for instance a methylene group. Typically, the hydrocarbon (main) chain includes 5, 6, 7 or 8 main chain atoms in each ring of the aromatic moiety. Examples of arylaliphatic moieties such as alkylaryl moieties include, but are not limited to, 1-ethyl-naphthalene, 1,1′-methylenebis-benzene, 9-isopropylanthracene, 1,2,3-trimethyl-benzene, 4-phenyl-2-buten-1-ol, 7-chloro-3-(1-methylethyl)-quinoline, 3-heptyl-furan 6-[2-(2,5-diethylphenyl)ethyl]-4-ethyl-quinazoline or, 7,8-dibutyl-5,6-diethyl-isoquinoline.

Each of the terms “aliphatic”, “alicyclic”, “aromatic” and “arylaliphatic” as used herein is meant to include both substituted and unsubstituted forms of the respective moiety. Substituents my be any functional group, as for example, but not limited to, amino, amido, azido, carbonyl, carboxyl, cyano, isocyano, dithiane, halogen, hydroxyl, nitro, organometal, organoboron, seleno, silyl, silano, sulfonyl, thio, thiocyano, trifluoromethyl sulfonyl, p-toluenesulfonyl, bromobenzenesulfonyl, nitrobenzenesulfonyl, and methanesulfonyl.

Representing the hollow silica particle by the symbol

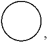

the entire particle with the polymer immobilized thereon can thus be depicted as:

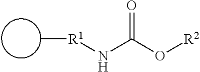

Typically the polymer defines a coating on the surface of the hollow silica particle. The polymer, R² in formula (1), may be any desired polymer. A respective polymer may for example be a homopolymer or a copolymer. The polymer may in some embodiments be a linear, i.e. straight, polymer. In some embodiments it may be a hyperbranched polymer. The polymer will usually be selected to have a molecular weight in the range from about 500-1000 000, such as about 500-500 000, 500-200 000, 500-100 000, 500-50 000, 500-25 000, 500-10 000 or 500-5000.

Generally, any polymer can be immobilized on a silica surface via the link defined above. As an illustrative example, the polymer may be a conducting polymer, such as polyaniline, polypyrrole and polythiophene. As a further example, the polymer may be an N-halamine polymer that shows biocidal activity and can be used as a disinfectant.

Typically the polymer is an amphiphilic or a hydrophilic, i.e. a polar, polymer. In some embodiments the polymer may nevertheless be at least essentially non-polar. As a further example, in embodiments where more than one polymer is immobilized on the particle, one of the polymers may be non-polar, while another polymer may be polar or amphiphilic. The term amphiphilic refers to a polymer that is soluble in both polar and non-polar fluids. The amphiphilic properties of the polymer are due to the presence of both polar and non-polar moieties within the same polymer. Polar properties of a respective polymer are based on polar moieties. Such moieties are for instance —COOH, —NH₂ or —OH side groups or terminal groups, in particular in the form of charged COO— or NH₃⁺ groups. In the form of side groups such groups may for example be carried by a polar, or in the case of an amphiphilic polymer for example by a non-polar backbone such as a hydrocarbon backbone of the polymer.

While ethers can—based on the dipole moment—be seen as only slightly polar, they have lone pairs of electrons on the oxygen atoms, allowing hydrogen bonding with for example water molecules. For sake of simplicity, ethers and polyethers as well as compounds, in particular polymers, with carbonyl groups are therefore, as used herein, regarded as polar and hydrophilic. Illustrative examples of respective polymers include, but are not limited to polymers that have a backbone with polar bonds such as poly(ethylene glycol), polyglycolic acid, polycaprolactone, polylactic acid or a polyhydroxyalkanoate, e.g. a polyhydroxybutyrate (poly-3-hydroxybutyrate, poly-4-hydroxybutyrate), poly-3-hydroxyvalerate, poly-3-hydroxypropionate, poly-2-hydroxybutyrate, poly-4-hydroxybutyrate, poly-4-hydroxyvalerate, poly-3-hydroxyhexanoate, poly-3-hydroxyheptanoate, poly-3-hydroxyoctanoate or poly-3-hydroxydecanoate. Illustrative examples of a polyhydroxyalkanoate copolymer include, but are not limited to, poly(3-hydroxybutyrate-co-3-hydroxyvalerate), poly(3-hydroxybutyrate-co-4-hydroxybutyrate), poly(3-hydroxybutyrate-co-3-hydroxyhexanoate), poly(4-hydroxybutyrate-co-3-hydroxyhexanoate), and poly(3-hydroxybutyrate-co-4-hydroxybutyrate-co-3-hydroxyhexanoate).

In some embodiments a respective hydrophilic or amphiphilic polymer is a biocompatible polymer, including a pharmaceutically acceptable polymer. The term “biocompatible polymer” (which also can be referred to as “tissue compatible polymer”), as used herein, is a polymer that produces little if any adverse biological response when used in vivo. The term thus generally refers to the inability of a polymer to promote a measurably adverse biological response in a cell, including in the body of an animal, including a human. A biocompatible polymer can have one or more of the following properties: non-toxic, non-mutagenic, non-allergenic, non-carcinogenic, and/or non-irritating. A biocompatible polymer, in the least, can be innocuous and tolerated by the respective cell and/or body. A biocompatible polymer, by itself, may also improve one or more functions in the body. A variety of biocompatible polymers is suitable for the formation of a microparticle or nanoparticle according to the invention. The biocompatible polymers can be synthetic polymers, naturally occurring polymers or combinations thereof. As used herein the term “synthetic polymer” refers to polymers that are not found in nature, including polymers that are made from naturally occurring biomaterials. Examples of suitable biocompatible polymers include non-absorbable polymers such as polypropylene, polyethylene, polyethylene terephthalate, polytetrafluoroethylene, polyaryletherketone, nylon, fluorinated ethylene propylene, polybutester, and silicone, or copolymers thereof (e.g., a copolymer of polypropylene and polyethylene); absorbable polymers such as polyglycolic acid (PGA), polylactic acid (PLA), polycaprolactone, and polyhydroxyalkanoate, copolymers thereof (e.g., a copolymer of PGA and PLA), and mixtures thereof.

A wide variety of biodegradable polymers is also suitable for a microparticle or nanoparticle that is water-soluble. Biodegradable polymers, as defined herein, are a subset of biocompatible polymers that gradually disintegrate or are absorbed in vivo over a period of time (e.g., within months or years). Disintegration may for instance occur via hydrolysis, may be catalysed by an enzyme and may be assisted by conditions to which the microparticles or nanoparticles are exposed in a human or animal body, including a tissue, a blood vessel or a cell thereof. Examples of biodegradable polymers suitable for a particle according to the invention include, but are not limited to, polyesters, polyanhydrides, polyorthoesters, polyphosphazenes, polyphosphates, polyphosphoesters, polyphosphonates, polydioxanones, polyhydroxyalkanoates, polycarbonates, polyalkylcarbonates, polyorthocarbonates, polyesteramides, polyamides, polyamines, polypeptides, polyurethanes, polyetheresters, or combinations thereof. An illustrative example of a biodegradable polymer is poly(α-hydroxy acid), for example polylactic acid, polyglycolic acid and copolymers and mixtures thereof such as poly(L-lactide) (PLLA), poly(D,L-lactide) (PLA); poly(glycolide) (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), poly(D,L-lactide-co-caprolactone) (PLA/PCL), poly(glycolide-co-caprolactone) (PGA/PCL); poly-ethylene oxide (PEO); polydioxanone (PDS); polypropylene fumarate; poly(ethyl glutamate-co-glutamic acid); poly(tert-butyloxy-carbonylmethyl glutamate); poly(carbonate-esters). Further examples of suitable biodegradable polymers include a polylacton such as a poly(ε-caprolactone) (PCL) and copolymers thereof such as polycaprolactone co-butylacrylate; polyhydroxybutyrate (PHBT) and copolymers of polyhydroxybutyrate; poly(phosphazene); poly(phosphate ester); a polypeptide; a polydepsipeptide, a maleic anhydride copolymer; a polyphosphazene; a polyiminocarbonate; poly(dimethyl-trimethylene carbonate-co-trimethylene carbonate); a polydioxanone, polyvalerolactone, a polyorthoester, a polyanhydride, polycyanoacrylate; a tyrosine-derived polycarbonate or polyester-amide; a polysaccharide such as hyaluronic acid; and copolymers and mixtures of the above polymers, among others. In some embodiments the biocompatible polymer may be crosslinked, for example to improve mechanical stability of the microparticle or nanoparticle. A further illustrative example of a suitable biocompatible polymer is a crosslinked dextrane polymer (Li et al., Angew. Chem. Int. Ed. (2009) 48, 52, 9914-9918).

As mentioned above, on the silica surface on which the polymer is immobilized, in the present case the surface of the hollow silica particle, the polymer has a particular high density. Accordingly, blank portions or patches of the respective surface—without polymer immobilized thereon—are found to a much lesser extent, if at all, than on polymer coated surfaces currently available in the art. The surface of the silica particle may for example have a polymer content/polymer density in the range from about 10% to about 90% (w/w), such as about 10% to about 80% (w/w), about 15% to about 90% (w/w), about 15% to about 80% (w/w), about 20% to about 90% (w/w), about 20% to about 80% (w/w), about 20% to about 70% (w/w), about 15% to about 70% (w/w) or about 20% to about 60% (w/w). Typically the surface has a polymer content/polymer density of at least about 20% (w/w), including at least about 25% (w/w), at least about 30% (w/w), at least about 35% (w/w), at least about 40% (w/w), or at least about 45% (w/w). In one embodiment the surface has a content of immobilized polymer of about 45% (w/w).

A particle according to the invention has typically a maximal width of about 1 nm to about 1 μm, such as about 1 nm to about 500 nm, about 2 nm to about 1 μm, about 2 nm to about 500 nm, about 2 nm to about 250 nm, about 10 nm to about 500 nm, about 10 nm to about 250 nm, about 25 nm to about 250 nm or about 50 nm to about 250 nm, such as about 250 nm, about 200 nm, about 190 nm or about 150 nm. The particle may be of any shape and geometry, including ball-shaped, i.e. spherical, rod shape, the shape of a disc or the shape of a rope.

The (in some embodiments water-soluble) microparticle or nanoparticle is typically a hollow micro- or nanosphere, i.e. it includes a void or cavity. In typical embodiments the microparticle or nanoparticle may have a shell of silica surrounding a void. The shell may be defined by a single wall with an internal and an external surface (i.e., balloon-like). The void or cavity may include the same fluid as an ambient fluid that surrounds the particle. The wall defining the shell may in some embodiments be considered to be an at least essentially closed and contiguous surface, in which some cracks and/or blowholes can occur. The wall defining the shell may in some embodiments have a thickness of about 0.1 nm to about 100 nm, such as about 1 nm to about 50 nm, about 2 nm to about 50 nm, about 5 nm to about 50 nm, about 2 nm to about 25 nm or about 5 nm to about 20 nm, such as about 10, 12, 14, 15 or about 20 nm.

In some embodiments the particle, for example within a shell thereof, has one or more pores via which the void or cavity is in fluid communication with the ambience. Accordingly, the particle may be microporous or mesoporous. Microporous matter is in the art understood to have pores of a width of less than about 2 nm, whereas mesoporous matter is understood as having pores of about 2 nm to about 50 nm. The particle may for instance have a porous shell with a pore width of about 0.1 nm to about 500 nm, including a pore width of about 0.1 nm to about 8 nm, of about 0.5 nm to about 6 nm, of about 1 nm to about 6 nm, of about 1 nm to about 10 nm, about 1 nm to about 50 nm, about 1 nm to about 100 nm, about 1 nm to about 250 nm, about 1 nm to about 500 nm, such as a pore width of about 1 nm. The pore volume of the particle is in some embodiments selected in the range from about 0.01 cm³/g to about 2 cm³/g, such as from about 0.05 cm³/g to about 2 cm³/g, from about 0.05 cm³/g to about 1 cm³/g, from about 0.1 cm³/g to about 1 cm³/g or from about 0.1 cm³/g to about 0.5 cm³/g, e.g. about 0.25 cm³/g. The particulate porous metal oxide or metalloid oxide has in some embodiments pore walls of at least about 1 nm, including at least about 2.5 nm. A respective mesoporous or microporous particle may have any form and shape, including a sphere, a rod, a disc or a rope. The pores may be arranged in an ordered arrangement with symmetry such as hexagonal, cubic or lamellar. As two illustrative examples, the particle may be of the M41 S family mesoporous silicas or an SBA type silica (SBA: Santa Barbara University), such as SBA-15 or SBA-16.

The pores of the particle can be analysed by a variety of techniques. Examples include, but are not limited to, transmission electron microscopy, scanning electron microscopy, gas, e.g. nitrogen, adsorption, inverse platinum replica imaging, small-angle X-ray scattering, small-angle neutron scattering and positron annihilation lifetime spectroscopy. In some embodiments of transmission electron microscopy (TEM) a series of TEM images is taken from the same position at different tilt angles and 3D-information obtained in the so called tomography mode. In some embodiments of scanning electron microscopy (SEM) high resolution-SEM is used, working at very low currents and voltages. Structural information can furthermore be taken from NMR, Raman and FTIR spectroscopies, electrochemical methods, UV-Vis absorption and fluorescence spectroscopies, as well as single molecule spectroscopic methods. In single molecule spectroscopic methods the materials are typically investigated by doping them with very low, usually nanomolar, concentrations of fluorescent dyes. Individual molecules and/or individual nanoscale environments can then be analysed.

The wall of the particle may be of small or even negligible thickness in comparison to the particle dimensions, such as less than about 10%, less than about 5%, less than about 2% or less than about 1% of the maximal particle width. The wall defining the shell may be meso- or microporous (supra, e.g. sponge-like) in nature. Further matter may be included in the respective void or cavity, such as a fluid, including a liquid. A respective fluid included within the particle may in some embodiments include or be a pharmaceutically active compound and/or an excipient. The pharmaceutically active compound may be a low molecular weight organic compound. In some embodiments the pharmaceutically active compound is or includes a peptide, a protein, a lipid, a saccharide or a polysaccharide. The pharmaceutically active compound may be more or less homogenously distributed, e.g. dispersed, within the water-soluble microparticle or nanoparticle. In some embodiments the pharmaceutically active compound is located within a certain portion of the water-soluble microparticle or nanoparticle, such as a core or a shell.

The particle may have a BET surface area in the range from about 10 m²/g to about 1000 m²/g, such as about 25 m²/g to about 500 m²/g, about 50 m²/g to about 250 m²/g, about 75 m²/g to about 200 m²/g or about 100 m²/g to about 200 m²/g, including a BET surface area of about 170 m²/g or about 160 m²/g.

On the external surface area, i.e. the surface area on the exterior side of a shell surface of a particle of the invention facing the ambience, the polymer is immobilized (supra). The external surface area of shell-based particles may be measured by means of techniques known to those skilled in the art such as Atomic Force Microscopy (AFM) and BET isotherm analysis. The external surface area of particles of the invention may range from about 10 to about 500 square meters/gram. The internal surface area, i.e. the surface area facing the interior side of a shell surface of particles of the invention, e.g. facing the void, is in contact with any matter that is included in such void of the particle, such as a pharmaceutically active compound. The internal surface area of shell-based particles with contiguous solid walls cannot be measured directly via techniques such as Atomic Force Microscopy (AFM) and BET isotherm analysis, but can be estimated based on the external particle surface area and particle wall thickness.

A silica particle according to the invention consists at least essentially of silica. The void of a hollow particle is understood not to be a part of the solid particle and accordingly to be excluded from the silica content of the particle, since it generally contains matter that differs from the particle as such. Typically the particle has a silica content of at least 90%, at least 94%, at least 95%, at least 96%, at least 97%, at least 98%, at least 98.5%, at least 99%, at least 99.5%, at least 99.8%, or at least 99.9%. Accordingly, the term “silica particle” for example includes a particle that is of silica and includes a dopant such as a metal, a metal oxide, a metal salt, a metalloid a salt of a metalloid, a metalloid oxide or a non-metal element or compound, e.g. nitrogen.

The method of covalently coupling a polymer to a silica surface may be applied to any silica surface. The silica surface may be the surface of any matter such as a silica coating or a device or structure of silica. The silica surface is in some embodiments planar. It may for instance be a straight, bend or contain additional geometric characteristics such as a dent or a bulge. In some embodiments the surface is at least essentially smooth. In some embodiments the surface is uneven. In some embodiments the silica surface is the surface of a nanoparticle or a microparticle. Such a nanoparticle or microparticle may include further matter such as a core of a metal or a mixture of metals. In some embodiments the particle is a silica particle. A respective silica particle may in some embodiments have a void and may be a hollow particle (supra). In some embodiments the particle is compact without a void.

The silica surface carries amino functional groups. Where the surface does not carry amino groups, amino groups can be introduced, for example via coupling of an amino-functional alkoxysilane or siloxane. A respective alkoxysilane may for example be of the general Formula (3):

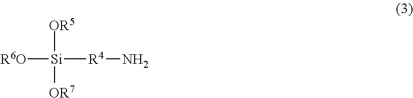

In Formula (3) R⁴ is an aliphatic, alicyclic, aromatic or arylaliphatic bridge with a main chain of 1 to about 30 carbon atoms such as 2 to about 30 carbon atoms or 2 to about 25 carbon atoms, including about 1 to about 20 carbon atoms, about 2 to about 20 carbon atoms, about 3 to about 20 carbon atoms, about 1 to about 15 carbon atoms, about 2 to about 15 carbon atoms, 1 to about 10 carbon atoms or about 2 to about 10 carbon atoms, such as 1, 2, 3, 4, 5, 6, 7, 8, 9, 10, 11, 12, 13, 14, 15; 16, 17, 18, 19 or 20 carbon atoms. The aliphatic bridge of R⁴ may include 0 to about 10 heteroatoms, such as 0 to about 9 heteroatoms, 0 to about 8 heteroatoms, 0 to about 7 or 0 to about 6, e.g. 0 to about 5 heteroatoms, such as 0 to about 4 heteroatoms, 0 to about 3 or 0 to about 2, e.g. 0, 1, 2, 3, 4 or 5 heteroatoms. These heteroatoms may be selected from the group N, O, S, Se and Si. R⁴ may for example be a straight or branched alkyl group. The term “alkyl” refers, unless otherwise stated, to a saturated aliphatic or alicyclic hydrocarbon chain, which may be straight or branched and include heteroatoms. The branches of the hydrocarbon chain may include linear chains as well as non-aromatic saturated cyclic elements. Illustrative examples of non-cyclic alkyl groups are methyl, ethyl, propyl, butyl, pentyl, hexyl, heptyl, octyl, nonyl, decyl, the n isomers of these radicals, isopropyl, isobutyl, isopentyl, neopentyl, sec.-butyl, tert.-butyl, neopentyl and 3,3-dimethylbutyl.

R⁵, R⁶ and R⁷ are, independent from one another, an aliphatic, alicyclic, aromatic aromatic or arylaliphatic group with a main chain of about 1 to about 10 carbon atoms such as 1, 2, 3, 4, 5, 6, 7, 8, 9 or 10 carbon atoms. The aliphatic, alicyclic, aromatic or arylaliphatic bridge of R⁴ may include 0 to about 5 heteroatoms, such as 0 to about 4 heteroatoms, 0 to about 3 or 0 to about 2, e.g. 0, 1, 2, 3, 4 or 5 heteroatoms. These heteroatoms may be selected from the group N, O, S, Se and Si.

The process of providing amino functional groups on the silica surface may be carried out at an elevated temperature, i.e. a temperature above ambient temperature. The temperature may for example be selected in the range from about 30° C. to about 150° C., about 30° C. to about 120° C., about 40° C. to about 100° C. or about 50° C. to about 100° C., e.g. about 50° C., about 60° C., about 70° C., about 80° C., about 90° C. or about 100° C. The process may also be carried out under an inert gas atmosphere, i.e. in the presence of gases that are not reactive, or at least not reactive to a detectable extent, with regard to the reagents and solvents used. Examples of a reactive inert atmosphere are nitrogen or a noble gas such as argon or helium.

The silica surface that carries amino functional groups is contacted with a polymer with a carbonate group of the general Formula (2):

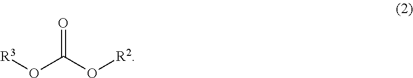

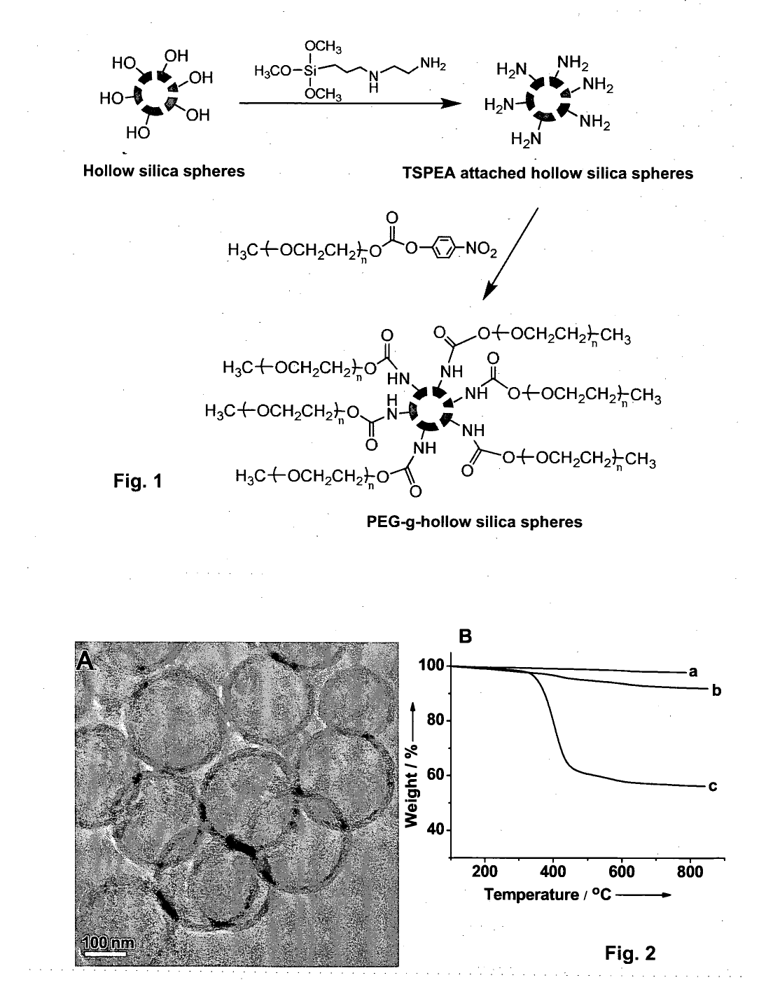

R² in general Formula (2) is the polymer (supra). R³ is an aliphatic, an alicyclic, an aromatic, an arylaliphatic group, or a silyl group. This group has a main chain of about 1 to about 20 carbon atoms, such as about 2 to about 20 carbon atoms, about 3 to about 20 carbon atoms, about 1 to about 15 carbon atoms, about 2 to about 15 carbon atoms, about 1 to about 10 carbon atoms, about 2 to about 10 carbon atoms or about 1 to about 10 carbon atoms, such as 1, 2, 3, 4, 5, 6, 7, 8, 9, 10, 11, 12, 13, 14, 15, 16, 17, 18, 19 or 20 carbon atoms. The main chain of R³ may include 0 to about 6 heteroatoms, such as 0 to about 5 heteroatoms, 0 to about 4 or 0 to about 2, including 0, 1, 2, 3, 4, 5 or 6 heteroatoms. These heteroatoms may be selected from N, O, S, Se and Si. A respective silyl group is an aliphatic, an alicyclic, an aromatic or an arylaliphatic group with a silicon atom defining the bridge, or the connection, to the carbonate moiety. The silicon atom generally carries further aliphatic, alicyclic, aromatic or arylaliphatic groups, which may in some embodiments be coupled to the silicon atom via an oxygen atom. Suitable examples of a group R³ include, but are not limited to, methyl-, ethyl-, propyl-, isopropyl-, butyl-, tert.-butyl, cyclobutyl-, 1-methylcyclobutyl-, allyl-, vinyl-, cinnamyl, 9-fluorenylmethyl, 2,2,2-trichloroethyl-, 2-trimethylsilylethyl-, 1,1-dimethylpropynyl-, 1-methyl-1-phenylethyl-, 1-methyl-1-(4-biphenylyl)ethyl-, 1,1-dimethyl-2-haloethyl-, 1,1-dimethyl-2-cyanoethyl-, 1-adamantyl-, cinnamyl-, 8-quinolyl-, N-hydroxypiperidinyl-, 2,4-dichlorobenzyl-, 5-benzisoxazolylmethyl-, diphenylmethyl-, isonicotinyl-, and S-benzyl-. As four further illustrative examples, R³ may be a nitrobenzyl- (e.g. para-nitrobenzyl- or 3,4-dimethoxy-6-nitrobenzyl-), a nitrophenyl-, a trifluoromethyl or a succinimde group. The aliphatic, alicyclic, aromatic, arylaliphatic or silyl group R³ can typically be taken to define a leaving group that is lost during the immobilization of the polymer to the silica surface. It is then replaced by the aliphatic, alicyclic, aromatic or arylaliphatic bridge R⁴ (supra) in a reaction that involves the terminal amino group, which is covalently linked to the bridge R⁴ (cf. FIG. 1 for an example, see also Formula (3) above).

The process of coupling a polymer to the silica surface may in some embodiments be carried out under an inert gas atmosphere (supra). The process may be carried out at an elevated temperature (supra). The temperature may for example be selected in the range from about 30° C. to about 120° C., about 40° C. to about 100° C. or about 50° C. to about 100° C., e.g. about 50° C.; about 60° C., about 70° C., about 80° C., about 90° C. or about 100° C. The process may also be carried out under an inert gas atmosphere, i.e. in the presence of gases that are not reactive, or at least not reactive to a detectable extent, with regard to the reagents and solvents used. Examples of a reactive inert atmosphere are nitrogen or a noble gas such as argon or helium. The process may take up to several days, such as about 24 hours, about 36 hours, about 48 hours, about 60 hours or about 72 hours.

The method of the invention is generally carried out in the liquid phase. It may be carried out in any suitable solvent. Any solvent may be used, as long as the polymer used dissolves therein sufficiently. Solvents used may be polar or non-polar liquids, including aprotic non-polar liquids. Examples of non-polar liquids include, but are not limited to, mineral oil, hexane, heptane, cyclohexane, benzene, toluene, pyridine, dichloromethane, chloroform, carbon tetrachloride, carbon disulfide and a non-polar ionic liquid. Examples of a non-polar ionic liquid include, but are not limited to, 1-ethyl-3-methylimidazolium bis[(trifluoromethyl)sulfonyl]amide bis(triflyl)amide, 1-ethyl-3-methylimidazolium bis[(trifluoromethyl)sulfonyl]amide trifluoroacetate, 1-butyl-3-methylimidazolium hexafluorophosphate, 1-hexyl-3-methylimidazolium bis(trifluoromethylsulfonyl)imide, 1-butyl-3-methylimidazolium bis(trifluoromethylsulfonyl)imide, trihexyl(tetradecyl)phosphonium bis[oxalato(2-)]borate, 1-hexyl-3-methyl imidazolium tris(pentafluoroethyl)trifluorophosphate, 1-butyl-3-methyl-imidazolium hexafluorophosphate, tris(pentafluoroethyl)trifluorophosphate, trihexyl(tetradecyl)phosphonium, N″-ethyl-N,N,N′,N′-tetramethylguanidinium, 1-butyl-1-methyl pyrroledinium tris(pentafluoroethyl)trifluorophosphate, 1-butyl-1-methyl pyrrolidinium bis(trifluoromethylsulfonyl)imide, 1-butyl-3-methyl imidazolium hexafluorophosphate, 1-ethyl-3-methylimidazolium bis(trifluoromethylsulfonyl)imide and 1-n-butyl-3-methylimidazolium.

In some embodiments the method is carried out in a polar solvent. Examples of a polar solvent include, but are not limited to, dioxane, diethyl ether, diisopropylether, ethylene glycol monobutyl ether, tetrahydrofuran, methyl propyl ketone, methyl isoamyl ketone, methyl isobutyl ketone, cyclohexanone, isobutyl isobutyrate, ethylene glycol diacetate, and a polar ionic liquid. Examples of a polar ionic liquid include, but are not limited to, 1-ethyl-3-methylimidazolium tetrafluoroborate, N-butyl-4-methylpyridinium tetrafluoroborate, 1,3-dialkylimidazolium-tetrafluoroborate, 1,3-dialkylimidazolium-hexafluoroborate, 1-ethyl-3-methylimidazolium bis(pentafluoroethyl)phosphinate, 1-butyl-3-methylimidazolium tetrakis-(3,5-bis(trifluoromethylphenyl)borate, tetrabutyl-ammonium bis(trifluoromethyl)imide, ethyl-3-methylimidazolium trifluoromethanesulfonate, 1-butyl-3-methylimidazolium methylsulfate, 1-n-butyl-3-methylimidazolium ([bmim]) octylsulfate, and 1-n-butyl-3-methylimidazolium tetrafluoroborate.

A polar protic solvent that may be used can be a solvent, that has, for example, a hydrogen atom bound to an oxygen as in a hydroxyl group or a nitrogen as in an amino group. More generally, any molecular solvent which contains dissociable H⁺, such as hydrogen fluoride, is called a protic solvent. The molecules of such solvents can donate an H⁺ (proton). Examples of polar protic solvents include, but are not limited to, water, an alcohol or a carboxylic acid. Examples of an alcohol include, but are not limited to, methanol, ethanol, 1,2-ethanediol (ethylene glycol), 1,3-propanediol (β-propylene glycol), 1,2-propanediol, n-propanol, iso-propanol, n-butanol, iso-butanol, tert-butanol, 2-butanol, 2,3-butanediol (dimethylethylene glycol), 2-methyl-1,3-propanediol, 1-pentanol (amyl alcohol), 2-pentanol, 2-methyl-3-butanol, 3-methyl-1-butanol (iso-pentanol), 3-pentanol (sec-amyl alcohol), 2,4-pentanediol (2,4-amylene glycol), 4-methyl-1,7-heptanediol, 1,9-nonanediol, cyclohexanol, propoxymethanol and 2-ethoxyethanol (ethylene glycol ethyl ether). As four illustrative examples of a carboxylic acid may serve acetic acid, propionic acid, valeric acid and caproic acid.

As mentioned above, the hollow particle of the invention may in some embodiments be water-soluble. Where desired, such a water-soluble microparticle or nanoparticle may be designed for sustained and for controlled delivery. In a sustained system the pharmaceutically active compound is delivered over a prolonged period of time, which overcomes the highly periodic nature of tissue levels associated with conventional (e.g. enteral or parenteral) administration of single doses of free compounds. The term ‘controlled’ indicates that control is exerted over the way in which the pharmaceutically active compound is delivered to the tissue(s) once it has been administrated to the organism to be treated, e.g. the patient.

Any pharmaceutically active compound may be included into the particle. In some embodiments such a compound is polar and water-soluble. In some embodiments the compound is amphiphilic. In some embodiments the compound is at least essentially non-polar and water-insoluble. The pharmaceutically active compound may be a low molecular weight organic compound. In some embodiments the pharmaceutically active compound is or includes a peptide, a protein, a peptoid, a lipid, a nucleic acid, a saccharide, an oligosaccharide, a polysaccharide or an inorganic molecule. The pharmaceutically active compound may be more or less homogenously distributed, e.g. dispersed, within the hollow microparticle or nanoparticle. In some embodiments the pharmaceutically active compound is located within a certain portion of the water-soluble microparticle or nanoparticle, such as a nanoparticle or the inner wall of a shell of the hollow particle. When provided in a hollow particle, compounds can at least partly be protected from the action of components of the ambience such as enzymes, e.g. proteases. In particular where the particle transiently passes a tissue or organ, e.g. the digestive tract, the particle thereby provides protection from degradation or modification. Nevertheless a compound may be provided in the hollow particle in the form of a prodrug if desired. Further, the particle can be used to direct the compound to a desired target or site of action by providing corresponding moieties on the surface of the particle. In such embodiments the application resembles rather a local than a systemic application. Using a particle to encompass a compound also allows the application of a compound that can otherwise hardly be applied via standard application routes, such as an at least essentially non-polar compound. In addition, depending on the selection of the pore size, the particle size and/or other structural features of the particle, the particle generally provides a diffusion barrier as well as a protection from flow and abrupt changes of the ambience. Therefore encompassing matter such as pharmaceutically active compounds in a hollow particle, e.g. a particle with pores, slows the release of matter therefrom. Accordingly, the half-life of compounds in the human or animal body can be controlled by selecting the structural properties of the particle. Typically the half-life of compounds in the human or animal body is longer when applied in a hollow particle, as compared to an application without using a particulate carrier.

As used herein, the term “prodrug” means a compound which is converted or released within the human or animal body, e.g. enzymatically, mechanically or electromagnetically, into its active form that has medical effects. A “prodrug” is accordingly a pharmacologically inactive derivative of a parent “drug” molecule. It requires spontaneous or enzymatic biotransformation within the physiological system of the human or animal to which it is administered. “Prodrugs” are commonly used in the art to overcome problems associated with stability, toxicity, lack of specificity, or limited bioavailability. They often offer advantages of solubility, tissue compatibility, or delayed release in the mammalian organism. As an illustrative example, a “prodrug” may be a low molecular weight compound with a protective group shielding a moiety or functional group thereof and thereby reversibly suppressing the activity of the functional group. A respective “prodrug” may become pharmaceutically active in vivo or in vitro when the protective group undergoes solvolysis or enzymatic removal. As a further illustrative example, a functional group may only be introduced into an active compound upon biochemical transformation such as oxidation, phosphorylation, or glycosylation. Thus a respective “prodrug” may only be converted into a pharmaceutically active compound by an enzyme, gastric acid, etc. in the human or animal body. The “prodrug” of a pharmaceutically active compound may be a hydrate or a non-hydrate. Common “prodrugs” include acid derivatives such as esters prepared by reaction of parent acids with a suitable alcohol (e.g., an aliphatic or alicyclic alcohol with a main chain of a length of up to about 10 carbon atoms), amides prepared by reaction of the parent acid compound with an amine, or basic groups reacted to form an acylated base derivative such as an aliphatic or alicyclic amide with a main chain of a length of up to about 10 carbon atoms.

In this regard the present invention also provides a pharmaceutical composition. The pharmaceutical composition includes one or more hollow particles that have a pharmaceutically active compound in the void of the hollow particle(s). As detailed above, the void may be encompassed by a shell that surrounds the void. The particle(s) that is/are included in the pharmaceutical composition are typically water-soluble. Generally a particle is rendered water-soluble by selecting an appropriate polar or amphiphilic polymer for immobilization thereon.

In some embodiments a respective particle, e.g. a water-soluble particle, of the invention is coupled to a molecule with binding affinity for a selected target tissue or for a selected target molecule, such as a microorganism, a virus particle, a peptide, a peptoid, a protein, a nucleic acid, a peptide, an oligosaccharide, a polysaccharide, an inorganic molecule, a synthetic polymer, a small organic molecule or a drug.

Illustrative examples of a molecule with binding affinity for a certain target are an antibody, a fragment thereof and a proteinaceous binding molecule with antibody-like functions. Examples of (recombinant) antibody fragments are Fab fragments, Fv fragments, single-chain Fv fragments (scFv), diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). An example of a proteinaceous binding molecule with antibody-like functions is a mutein based on a polypeptide of the lipocalin family (WO 03/029462, Beste et al., Proc. Natl. Acad. Sci. U.S.A. (1999) 96, 1898-1903). Lipocalins, such as the bilin binding protein, the human neutrophil gelatinase-associated lipocalin, human Apolipoprotein D or glycodelin, possess natural ligand-binding sites that can be modified so that they bind to selected small protein regions known as haptens. Examples of other proteinaceous binding molecules are the so-called glubodies (see e.g. internation patent application WO 96/23879), proteins based on the ankyrin scaffold (Mosavi, L. K., et al., Protein Science (2004) 13, 6, 1435-1448) or crystalline scaffold (e.g. international patent application WO 01/04144) the proteins described in Skerra, J. Mol. Recognit. (2000) 13, 167-187, AdNectins, tetranectins and avimers. Avimers contain so called A-domains that occur as strings of multiple domains in several cell surface receptors (Silverman, J., et al., Nature Biotechnology (2005) 23, 1556-1561). Adnectins, derived from a domain of human fibronectin, contain three loops that can be engineered for immunoglobulin-like binding to targets (Gill, D. S. & Damle, N. K., Current Opinion in Biotechnology (2006) 17, 653-658). Tetranectins, derived from the respective human homotrimeric protein, likewise contain loop regions in a C-type lectin domain that can be engineered for desired binding (ibid.). Peptoids, which can act as protein ligands, are oligo(N-alkyl) glycines that differ from peptides in that the side chain is connected to the amide nitrogen rather than the a carbon atom. Peptoids are typically resistant to proteases and other modifying enzymes and can have a much higher cell permeability than peptides (see e.g. Kwon, Y.-U., and Kodadek, T., J. Am. Chem. Soc. (2007) 129, 1508-1509).

An example of a nucleic acid molecule with antibody-like functions is an aptamer. An aptamer folds into a defined three-dimensional motif and shows high affinity for a given target structure. Using standard techniques of the art such as solid-phase synthesis an aptamer with affinity to a certain target can accordingly be formed and immobilized on a hollow particle of the invention.

As a further illustrative example, a linking moiety such as an affinity tag may be used to immobilise a molecule with binding affinity for a selected target tissue or for a selected target molecule. Such a linking moiety may be a molecule, e.g. a hydrocarbon-based (including polymeric) molecule that includes nitrogen-, phosphorus-, sulphur-, carben-, halogen- or pseudohalogen groups, or a portion thereof. As an illustrative example, the silica surface may include functional groups. Such functional groups may be residual groups, e.g. amino groups, intended to be used and/or provided for the covalent attachment of the polymer, and which did not undergo a coupling reaction therewith. These groups may allow for the covalent attachment of a biomolecule, for example a molecule such as a protein, a nucleic acid molecule, a polysaccharide or any combination thereof.

Examples of an affinity tag include, but are not limited to, biotin, dinitrophenol or digoxigenin, oligohistidine, polyhistidine, an immunoglobulin domain, maltose-binding protein, glutathione-S-transferase (GST), calmodulin binding peptide (CBP), FLAG′-peptide, the T7 epitope (Ala-Ser-Met-Thr-Gly-Gly-Gln-Gln-Met-Gly), maltose binding protein (MBP), the HSV epitope of the sequence Gln-Pro-Glu-Leu-Ala-Pro-Glu-Asp-Pro-Glu-Asp of herpes simplex virus glycoprotein D, the hemagglutinin (HA) epitope of the sequence Tyr-Pro-Tyr-Asp-Val-Pro-Asp-Tyr-Ala, the “myc” epitope of the transcription factor c-myc of the sequence Glu-Gln-Lys-Leu-Ile-Ser-Glu-Glu-Asp-Leu, or an oligonucleotide tag. Such an oligonucleotide tag may for instance be used to hybridise to an immobilised oligonucleotide with a complementary sequence. A further example of a linking moiety is an antibody, a fragment thereof or a proteinaceous binding molecule with antibody-like functions (see also above).

A further example of a linking moiety is a cucurbituril or a moiety capable of forming a complex with a cucurbituril. A cucurbituril is a macrocyclic compound that includes glycoluril units, typically self-assembled from an acid catalyzed condensation reaction of glycoluril and formaldehyde. A cucurbit[n]uril, (CB[n]), that includes n glycoluril units, typically has two portals with polar ureido carbonyl groups. Via these ureido carbonyl groups cucurbiturils can bind ions and molecules of interest. As an illustrative example cucurbit[7]uril (CB[7]) can form a strong complex with ferrocenemethylammonium or adamantylammonium ions. Either the cucurbit[7]uril or e.g. ferrocenemethylammonium may be attached to a biomolecule, while the remaining binding partner (e.g. ferrocenemethylammonium or cucurbit[7]uril respectively) can be bound to a selected surface. Contacting the biomolecule with the surface will then lead to an immobilisation of the biomolecule. Functionalised CB[7] units bound to a gold surface via alkanethiolates have for instance been shown to cause an immobilisation of a protein carrying a ferrocenemethylammonium unit (Hwang, I., et al., J. Am. Chem. Soc. (2007) 129, 4170-4171).

Further examples of a linking moiety include, but are not limited to an oligosaccharide, an oligopeptide, biotin, dinitrophenol, digoxigenin and a metal chelator (cf. also below). As an illustrative example, a respective metal chelator, such as ethylenediamine, ethylenediaminetetraacetic acid (EDTA), ethylene glycol tetraacetic acid (EGTA), diethylenetriaminepentaacetic acid (DTPA), N,N-bis(carboxymethyl)glycine (also called nitrilotriacetic acid, NTA), 1,2-bis(o-aminophenoxy)ethane-N,N,N′,N′-tetraacetic acid (BAPTA), 2,3-dimercapto-1-propanol (dimercaprol), porphine or heme may be used in cases where the target molecule is a metal ion. As an example, EDTA forms a complex with most monovalent, divalent, trivalent and tetravalent metal ions, such as e.g. silver (Ag⁺), calcium (Ca²⁺), manganese (Mn²⁺), copper (Cu²⁺), iron (Fe²⁺), cobalt (Co³⁺) and zirconium (Zr⁴⁺), while BAPTA is specific for Ca²⁺. In some embodiments a respective metal chelator in a complex with a respective metal ion or metal ions defines the linking moiety. Such a complex is for example a receptor molecule for a peptide of a defined sequence, which may also be included in a protein. As an illustrative example, a standard method used in the art is the formation of a complex between an oligohistidine tag and copper (Cu²⁺), nickel (Ni²⁺), cobalt (Co²⁺), or zink (Zn²⁺) ions, which are presented by means of the chelator nitrilotriacetic acid (NTA). Avidin or streptavidin may for instance be employed to immobilise a biotinylated nucleic acid, or a biotin containing monolayer of gold may be employed (Shumaker-Parry, J. S., et al., Anal. Chem. (2004) 76, 918).

A molecule or moiety immobilized on a plurality of hollow particles may also serve in cross-linking individual particles. An organic bridge may for instance be formed between two particles by a phenylene-bridge or an ethylene-bridge. The bridge may also include a chiral moiety such as bulk vanadyl Schiff base complexes, a binaphthyl group, a 1,2-diaminocyclohexane group, a trans-(1R,2R)-bis-(ureido)-cyclohexyl-bridge (Zhu, G., et al., Microporous and Mesoporous Materials (2008) 116, 36-43) or a chiral borated ethylene-bridge. The resulting particulate matter is then a chiral porous material with chiral induction ability in e.g. asymmetric catalysis. A further example of a chiral moiety that may be immobilized on a hollow particle is a carbohydrate, including a cellulose derivative such as cellulose tris(3,5-dimethylphenyl carbamate).

A hollow silica particle according to the present invention may be used as a catalyst or as a support for a catalyst. In some embodiments it may for instance be used in catalytic combustion, such as the oxidation of volatile organic compounds, e.g. propen (Orlov & Klinowski, 2009, supra). A hollow silica particle according to the invention may also be functionalized with chelating ligands, to which catalytically active organometallic complexes may be complexed. As an illustrative example, diphenylphosphino ligands may be covalently bound to the surface, including the polymer thereon, of the hollow silica particle, and Pd- or Ru-containing organometallic silanes chelated thereto as described by Zhang et al. (Advanced Functional Materials (2008) 18, 3590-3597). Multiple active sites may be introduced, thereby forming a multifunctional catalyst (ibid.).

The present invention also relates to the use of hollow silica particles in e.g. the separation of a mixture of molecules in a fluid, in catalysis, in nonlinear optics, as an ion-exchange coating, in the formation of a solid-state electrochemical device and in the formation of a drug delivery vehicle. Since silica has high biocompatibility and at the same time mechanical strength, thermal and pH stability, and a large variety of polymers can be immobilized on the surface, a suitable polymer can be chosen for practically any intended purpose.

In some embodiments micro- or nanoparticles of the invention can be used in the separation of a mixture of molecules in a fluid such as chromatography, e.g. as gas chromatography, capillary electrochromatography, HPLC (high performance liquid chromatography) or UPLC (ultrahigh pressure liquid chromatography). In such separation applications a plurality of the hollow micro- or nanoparticles is typically used as a chromatography stationary phase.

As indicated above, particles according to the invention can be used as a carrier for a drug, a marker or other matter to be administered to a human or animal body. The micro- or nanoparticles described herein, as well as matter such as compounds included therein, can be administered to a cell, an animal or a human patient per se, or in a pharmaceutical composition. In a pharmaceutical composition the particles may be mixed with other active ingredients, as in combination therapy, or with suitable carriers or excipient(s). Techniques for formulation and administration of respective particles resemble or are identical to those of low molecular weight compounds well established in the art. Exemplary routes include, but are not limited to, oral, transdermal, and parenteral delivery. A plurality of the particles may be used to fill a capsule or tube, or may be compressed as a pellet. The micro- or nanoparticles may also be used in injectable or sprayable form, for instance as a suspension or in a gel formulation.

Suitable routes of administration may, for example, include depot, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections. It is noted in this regard that for administering micro- or nanoparticles a surgical procedure is not required. Where the micro- or nanoparticles include a biodegradable polymer there is no need for device removal after release of the anti-cancer agent. Nevertheless the particles may be included in or on a scaffold, a coating, a patch, composite material, a gel or a plaster.

In some embodiments one may administer the particles in a local rather than systemic manner, for example, via injection of the compound directly into a solid tumor, often in a depot or sustained release formulation.

Pharmaceutical compositions that include the particles of the present invention may be manufactured in a manner that is itself known, e. g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers including excipients and auxiliaries that facilitate processing of the particles into preparations that can be used pharmaceutically. Proper formulation is dependent upon the route of administration chosen.

For injection, the particles of the invention may be formulated in aqueous solutions, for instance in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the micro- or nanoparticles can be formulated readily by combining them with pharmaceutically acceptable carriers well known in the art. Such carriers enable the particles, including a compound included therein to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatine, as well as soft, sealed capsules made of gelatine and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the particles may be suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

The micro- or nanoparticles may be formulated for parenteral administration by injection, e.g., by bolus injection or continuous infusion. Formulations for injection may be presented in unit dosage form, e. g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical compositions suitable for use in the present invention include compositions where the active ingredients included in the micro- or nanoparticles are contained in an amount effective to achieve its intended purpose. More specifically, a therapeutically effective amount means an amount of a compound effective to prevent, alleviate or ameliorate cancer or prolong the survival of the subject being treated. Determination of a therapeutically effective amount is well within the capability of those skilled in the art.

For any compound used in the micro- or nanoparticles of the invention, the therapeutically effective dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the kinase activity). Such information can be used to more accurately determine useful doses in humans.

The micro- or nanoparticles may, if desired, be presented in a pack or dispenser device which may contain one or more unit dosage forms containing the particles with the active ingredient. The pack may for instance include metal or plastic foil, such as a blister pack. The pack or dispenser device may be accompanied by instructions for administration. The pack or dispenser may also be accompanied with a notice associated with the container in a form prescribed by a governmental agency regulating the manufacture, use, or sale of pharmaceuticals, which notice is reflective of approval by the agency of the form of the compound for human or veterinary administration. Such notice, for example, may be the labeling approved by the U. S. Food and Drug Administration or other government agency for prescription drugs, or the approved product insert.

In order that the invention may be readily understood and put into practical effect, particular embodiments will now be described by way of the following non-limiting examples. It is understood that modification of detail may be made without departing from the scope of the invention.

### Exemplary Embodiments of the Invention

Exemplary embodiments of methods according to the invention as well as reactants and further processes that may be used are shown in the appending figures.

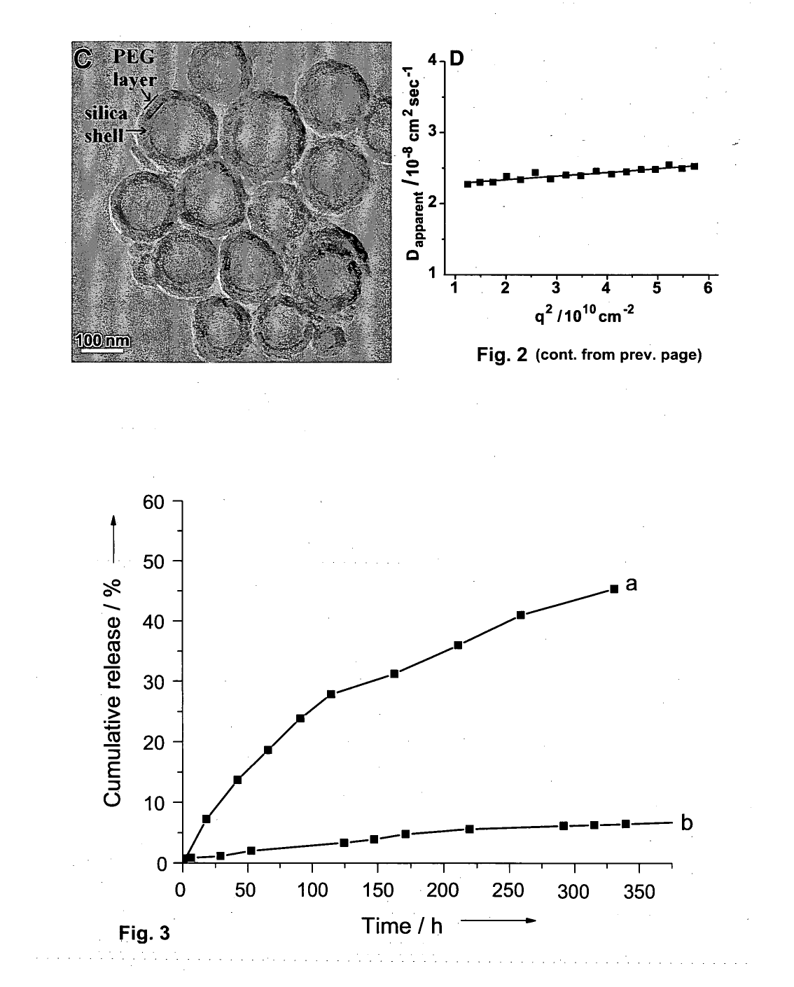

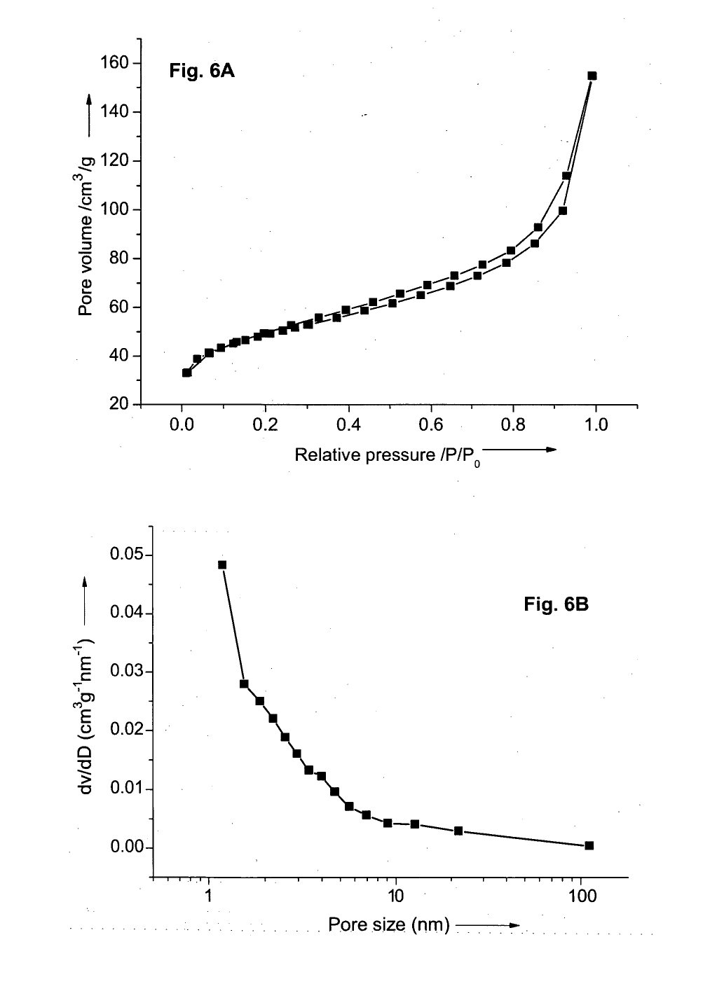

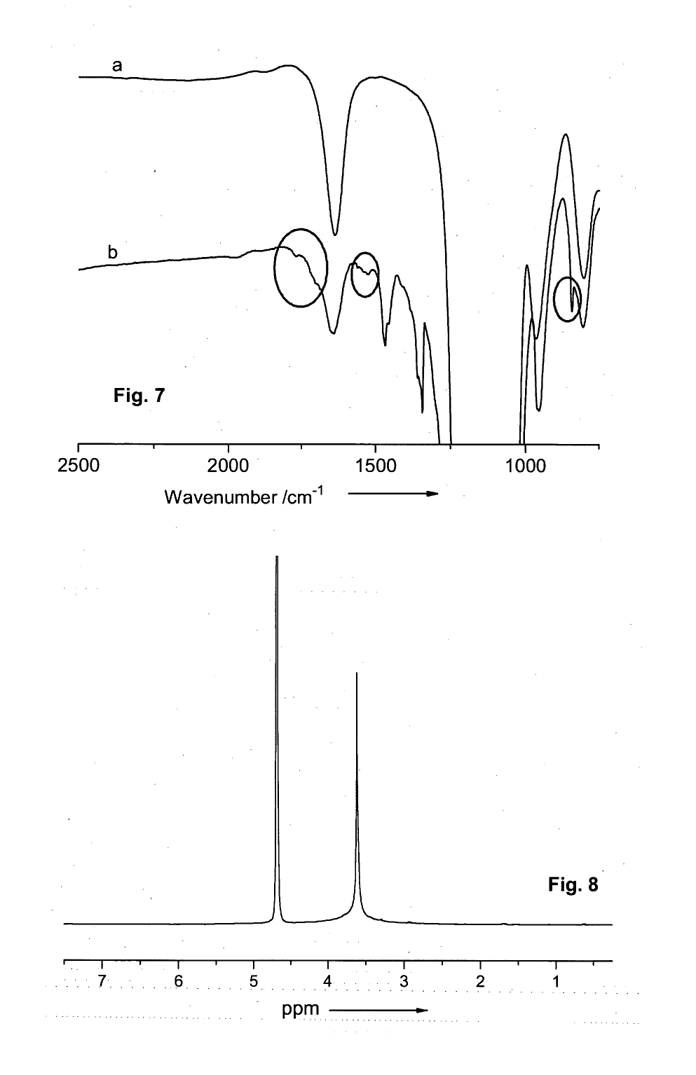

In the Examples, PEG-g-hollow silica vesicles are obtained by grafting PEG brushes to nanosized porous hollow silica spheres. To prepare PEG-g-hollow silica vesicles (as shown in FIG. 1), first nanosized hollow silica spheres were prepared by sol-gel reaction of TEOS in the presence of polystyrene templates followed by removing polystyrene templates under calcination at 550° C. (Bamnolker, H. et. Al., J. Mater. Sci. Lett. (1997) 16, 1412-1415; Deng, Z W, et al., Langmuir (2006) 22, 6403-6407). From TEM images shown in FIG. 2A, the average diameter and the average shell thickness of the hollow silica spheres are determined to be ca. 190 nm and ca. 14 nm respectively. The hollow silica spheres have pores with an average diameter of 1.19 nm, a surface area of 164 m²/g and a total pore volume of 0.24 cm³/g which were measured using BET and BJH methods (FIG. 6A & FIG. 6). Then hollow silica spheres were modified with N-(3-trimethoxysilyl)propyl ethylene diamines (TMSPEA). As indicated by TGA results shown in FIG. 2B, the degradation degree of TMSPEA attached hollow silica spheres increases by 6% (w/w) at 750° C. as compared with the pristine hollow silica spheres. Finally PEG-g-hollow silica vesicles were obtained by grafting PEG brushes via urethane groups formed by the reaction among the amino groups on the surfaces of TMSPEA attached hollow silica spheres and monomethoxy PEG (Mn: 5 K) 4-nitrophenyl carbonate. The successful PEG grafting is verified by the characteristic peaks of urethane group at 1769, 1701 1526, 842 cm⁻¹ observed in FTIR spectrum of PEG-g-hollow silica vesicles (FIG. 7); and the peaks at 3.61 ppm in ¹H NMR spectrum (FIG. 8) attributed to the protons of PEG. From the thermogravimetric (TGA) curve of PEG-g-hollow silica vesicles shown in FIG. 2B (c), the content of PEG in PEG-g-hollow silica vesicles is ca. 210 nm and 37 nm respectively.

The grafted PEG stealth layers render PEG-g-hollow silica vesicles good dispersion in aqueous solution. Dynamic laser light scattering (DLLS) was applied to characterize the aqueous solution of PEG-g-hollow silica vesicles. FIG. 1D shows that the apparent diffusion coefficient, Dₐₚₚ, of PEG-g-hollow silica vesicles obtained from DLLS measurements is almost independent of the square of the wave scatter vector, q². This indicates the spherical shape of species in solution (Du, J Z, et al., J. Am. Chem. Soc. (2003) 125, 14710-14711). The hydrodynamic diameter, Rₕ, of PEG-g-hollow silica vesicles is 205 nm (with a polydispersity index (PDI) of 0.075) which is close to the average diameter of the individual vesicles obtained from TEM images. So PEG-g-hollow silica vesicles are dispersed well in aqueous solution without aggregation. Significant dilution of the aqueous solution even to a concentration of 1 mg in thousands mL did not affect the Rₕ value of PEG-g-hollow silica vesicles, and the vesicles have a long shelf-life as reflected by the consistent Rₕ value of vesicles in more than 12 months in aqueous solution.

Silica is biodegradable in vivo (Barbe, C, et al., Adv. Mater. (2004) 16, 1959-1966), so PEG-g-hollow silica vesicles should have a good biocompatibility. All these features make PEG-g-Hollow silica vesicles promising for drug delivery. Here PEG-g-hollow silica vesicles are explored for delivery of water insoluble drug paclitaxel (PTX). PTX is a potent drug for chemotherapy of several types of cancer but has a very low solubility in aqueous solution and acute adverse effects (Rowinsky, E K, et al., New Engl. J. Med. (1995) 332, 1004-1014). Therefore a suitable formulation is necessary to improve its bioavailability and reduce its side effects. The formulation of PTX using Cremophor-EL is in clinic uses, but Cremophor-EL can induce hypersensitivity and other side effects (Gelderblom, H., et al., Eur. J. Cancer (2001) 37, 1590-1598; Singla, A K, et al., Int.l J. Pharm. (2002) 235, 179-192). Although formulation of PTX without Cremophor EL has been pursued including using liposomes (Crosasso, P, et al., J. Control. Release (2000) 63, 19-30), polymer micelles (Shuai, X T, et al., Bioconjugate Chem. (2004) 15, 441-448) and polymer nanoparticles (Dong, Y, Feng, S S, Biomaterials (2007) 28, 4154-4160), better formulations are still desirable. To load PTX, PEG-g-hollow silica vesicles were immersed in a saturated methanol solution of PTX. Then PEG-g-hollow silica vesicles filled with saturated methanol solution of PTX were collected by centrifugation followed by drying. The solid obtained was dispersed in aqueous solution and the solution was filtered through membranes with pores of 5 μm diameter to remove free PTX, and the filtrate was lyophilized to get PTX loaded PEG-g-hollow silica (in a controlled experiment, free PTX can be removed by the filtration). The PTX content in PTX loaded PEG-g-hollow silica vesicles was determined to be ca. 5% using HPLC.

It was reported that no PTX can be adsorbed onto silica surfaces in methanol due to the strong interaction among PTX molecules and methanol molecules (Hata, H, et al., Chem. Mater. (1999) 11, 1110-1119). Therefore, PTX was just loaded in the interiors and pores of PEG-g-hollow silica vesicles. For this simple filling model, the PTX content (PTX % (w/w)) can be calculated using equation 1:

$$PTX \% ( w / w ) = \frac{{V}_{pore} + {V}_{interior}}{{W}_{silica} + {W}_{PEG}} \times S \times 100 \% \qquad ( 1 )$$

where Vₚₒᵣₑ and Vᵢₙₜₑᵣᵢₒᵣ are the volume of the pores and the interior of hollow silica spheres, and W_{silica} and W_{PEG} are the weight of the silica shells and PEG stealth layers of PEG-g-hollow silica vesicles, respectively, and S is the solubility of PTX in methanol which was determined to be 50 mg/mL. From the BET, TEM and TGA results, the theoretical value of PTX % (w/w) is calculated to be ca. 4.6% (the detailed procedure is listed below), which agrees with the experimental result well. This verifies that PTX is held in the pores and interiors of PEG-g-hollow silica vesicles physically. PTX loaded PEG-g-hollow silica vesicles still can be dispersed in aqueous solution well with Rₕ of PTX loaded PEG-g-hollow silica being ca. 230 nm with a PDI of 0.136 as determined using DLLS. PTX loaded in PEG-g-hollow silica vesicles is stable after being stored at 4° C. for more than 12 months as indicated by HPLC results.

The release profile of PTX from PEG-g-hollow silica vesicles is shown in FIG. 2(a). 47% of the loaded PTX was released after 330 h. In comparison, only 5% of PTX from free PTX was released as shown in FIG. 2(b). Free PTX is in a form of particles of diameter higher than 5 μm (because a filtration through a membrane with pore diameter of 5 μm can remove all the PTX dispersed in aqueous solution). In contrast, PTX loaded in PEG-g-hollow silica vesicles is nanosized with a much higher surface area, hence PTX can be released at a higher rate.

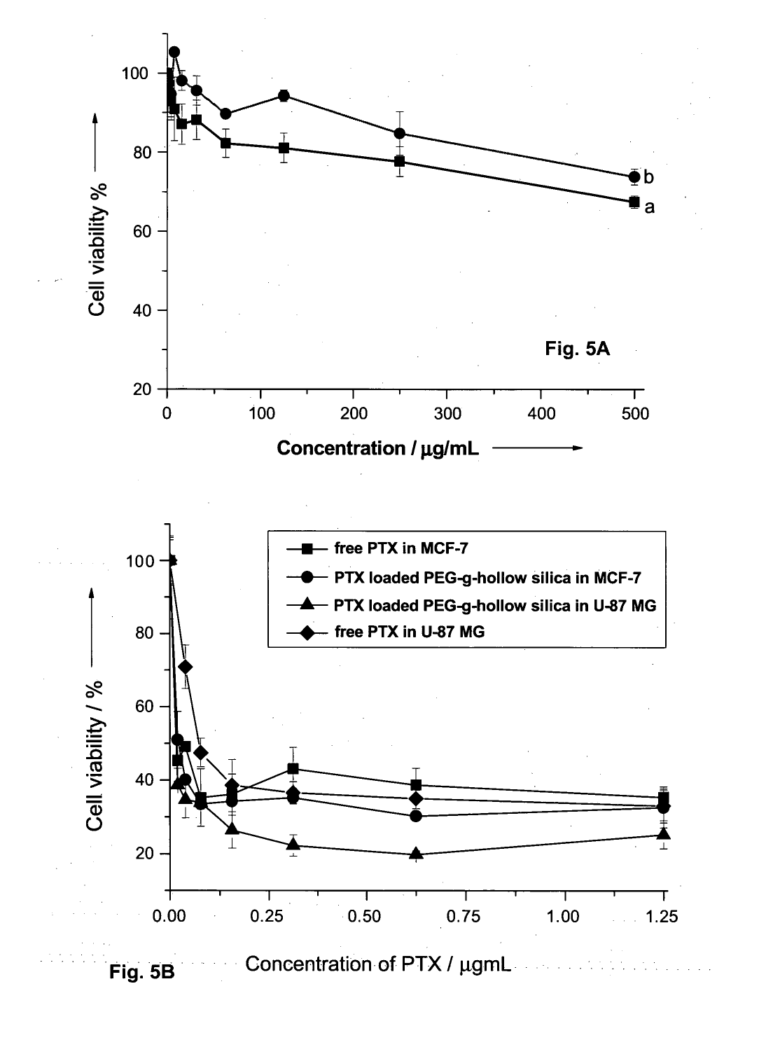

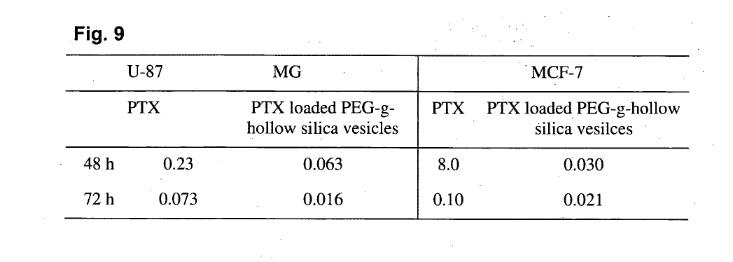

In vitro cytotoxicity of PEG-g-hollow silica vesicles and the efficacy of PTX loaded PEG-g-hollow silica vesicles to kill cancer cells were evaluated in MCF-7 cells and U-87 MG cells. In both cells, PEG-g-hollow silica vesicles show a low cytotoxicity. As shown in FIG. 5A, more than 65% of cells are still viable even when the concentration of PEG-g-hollow silica vesicles is up to 500 μg/mL after incubation for 72 h. It is reasonable because PEG and silica are well recognized to be biocompatible materials with low cytotoxicity. However, PTX loaded PEG-g-hollow silica vesicles are potent to kill cancer cells. FIG. 5B shows in vitro cytotoxicity of PTX loaded PEG-g-hollow silica vesicles in MCF-7 cells and U-87 MG cells after incubation for 72 h. After incubation for 72 h in MCF-7 cells, IC₅₀ of PTX loaded PEG-g-hollow silica vesicles is 0.021 μg/mL as compared to 0.10 μg/mL for free PTX. Similar results were observed in U-87 MG cells. After incubation for 72 h, IC₅₀ of PTX loaded PEG-g-hollow silica vesicles is 0.016 μg/mL as compared to 0.073 μg/mL for free PTX. IC₅₀ values of PTX loaded PEG-g-hollow silica vesicles in MCF-7 cells and U-87 MG cells after incubation for 48 h and 72 h are summarized in FIG. 9. Generally the IC₅₀ of PTX loaded PEG-g-hollow silica vesicles is lower than that of free PTX. Hence PTX loaded in PEG-g-hollow silica vesicles is potent to kill cancer cells.

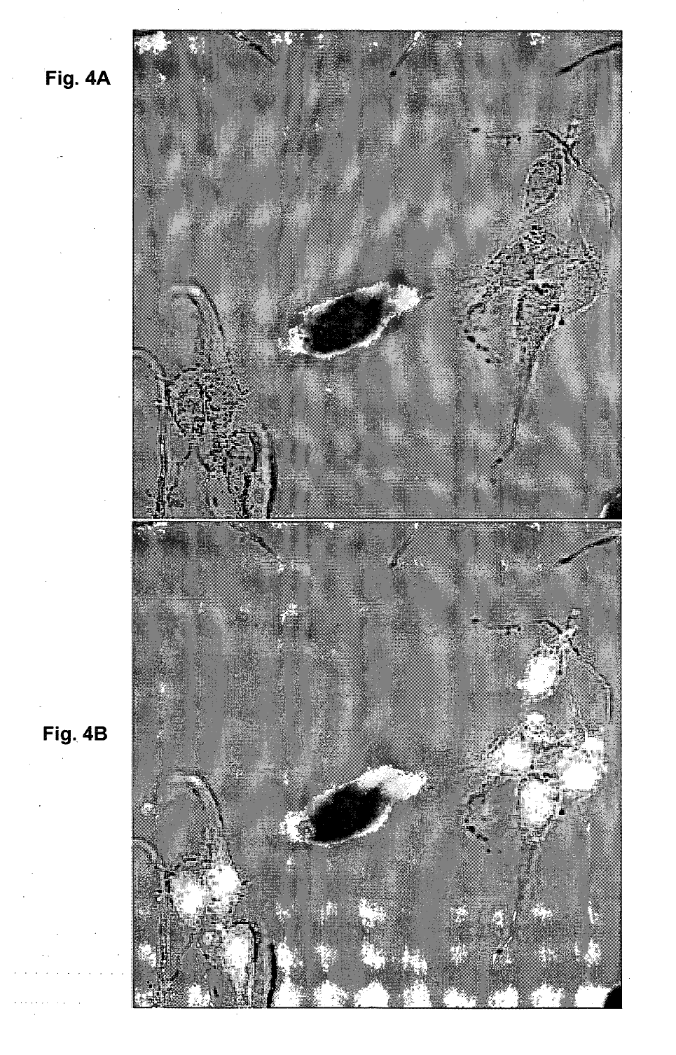

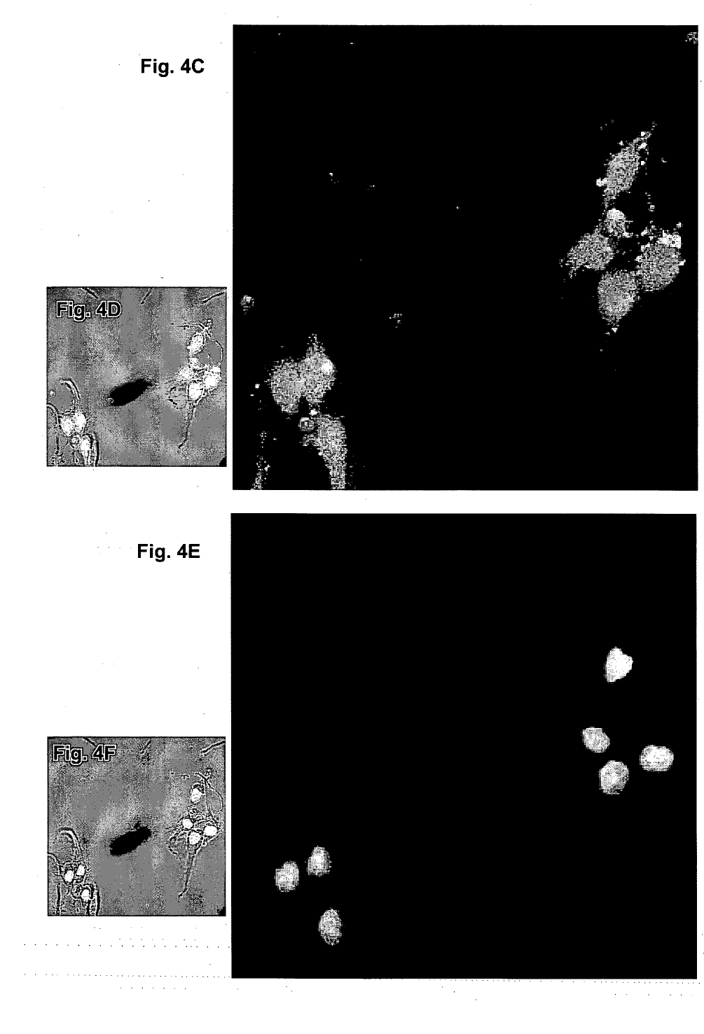

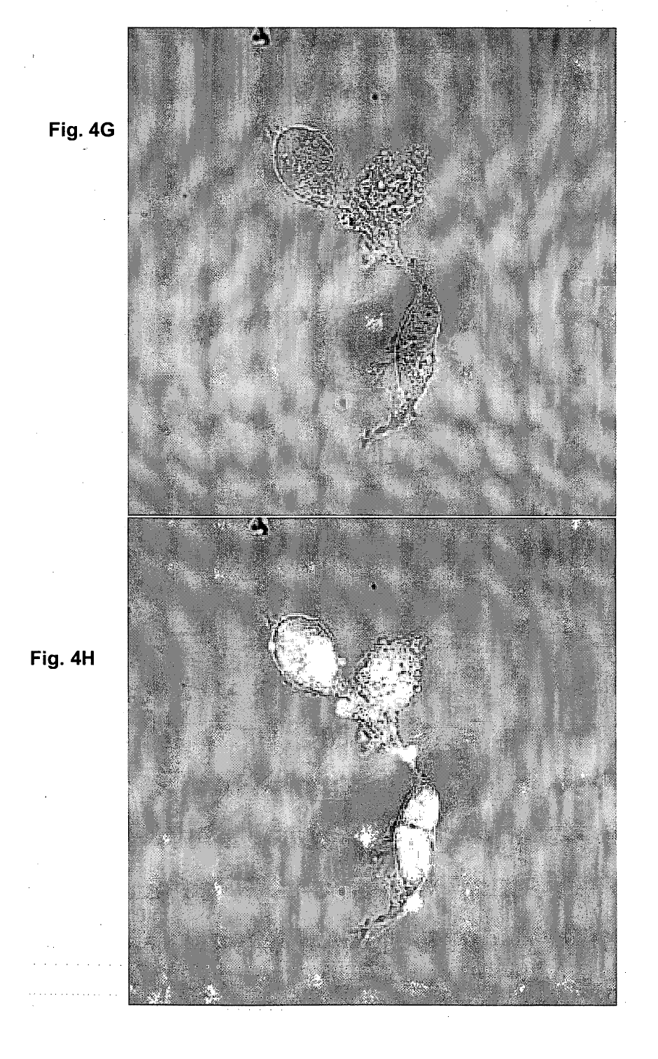

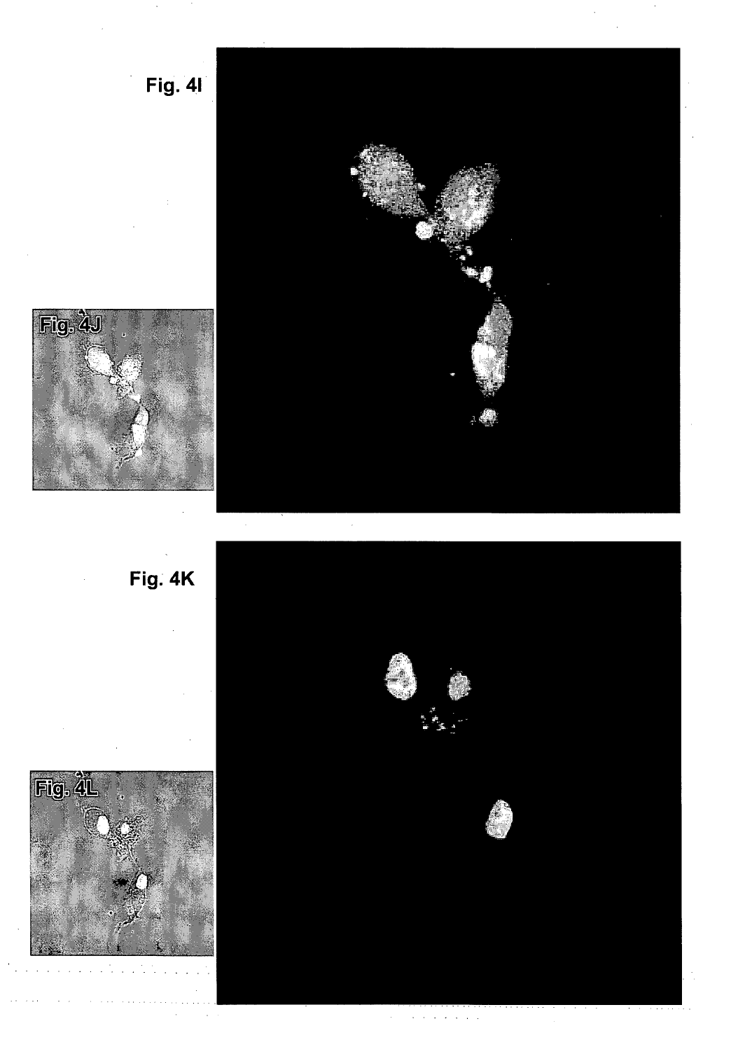

In order to understand the interaction of PEG-g-hollow silica vesicles with cells, FITC labelled PEG-g-hollow silica vesicles were prepared and were incubated with MCF-7 cells and U-87 MG cells for 18 h. After staining cellular nuclei with DAPI, confocal fluorescence micrographs were obtained as shown in FIG. 4. It is obvious that green FTIC labelled PEG-g-hollow silica vesicles can be observed in MCF-7 cells and U-87 MG cells. So PEG-g-hollow silica vesicles can be internalized by the cells. This could improve the bioavailability of PTX loaded in PEG-g-hollow silica vesicles together with the capability to release PTX fast. PTX leads to disassembly of microtubules in cells which cause dysfunction of microtubules and death of cell (Rowinsky, E K, & Donehower, R C, New Engl. J. Med. (1995) 332, 1004-1014). Therefore PTX loaded PEG-g-hollow silica vesicles could have a good efficacy to kill cancer, cells.

### Materials

Styrene from Merck was stored at 4° C. after distillation to remove the inhibitor. HPLC grade water, p-xylene (anhydrous, 99%), monomethyl poly(ethylene glycol) (PEG) (MW of 5K), N-[3-(trimethoxysilyl)]propyl ethylene diamines (TMSPEA) (97%), and dimethyl sulfoxide (DMSO) (99.9%) were from Sigma Aldrich. α,α′-azodiisobutyramidine dihydrochloride (AIBA) (>98%), 4-nitropheyl chloroformate (99%), triethylamine (>98), and poly(vinyl-pyrrolidone) (PVP) with molecular weight of 40,000 were supplied by Fluka. Absolute ethanol and tetraethoxysilane (99.999%) were purchased from Merck. Ammonia hydroxide (NH₄OH) (25 wt %) were from Honeywell. Diethyl ether, acetonitrile, and chloroform were supplied by J. T. Baker. Paclitaxel (PTX) (purity>99%) was purchased from Yunnan Hande Bio-Tech Co. Ltd (Kunming, China). Phosphate buffered saline (PBS) was obtained from NUMI Media Preparation Facility (National University of Singapore). Thiazolyl blue [3-(4,5-dimethyliazolyl-2)-2,5-diphenyl tetrazolium bromide] (MTT), dubelcco's modified Eagle's minimal essential medium (DMEM), fetal bovine serum (FBS), penicillin-streptomycin and trypsin-EDTA were obtained from Invitrogen. All chemicals excluding styrene were used as received. MCF-7 breast cancer cells and U-87 MG brain cancer cells were obtained from American type Culture Collection (ATCC).

### Characterization

TEM observations were performed with a Philips CM300 FEGTEM instrument at 300 KV. The samples were prepared by dipping copper meshes covered with carbon into aqueous solution of samples followed by drying in air.

Thermogravimetric analysis (TGA) analyses were carried out on a Thermal Analysis TGA 2050 under N₂ with a flow rate of 60 mL/min and a heating rate of 15° C./min from 100° C. to 850° C. Nitrogen adsorption-desorption isotherm measurements were performed on a Quantachrome Nova 1000 series BET surface analyzer at 77 K under a continuous adsorption condition. Hollow silica particles were degassed under vacuum at 200° C. before measurements. Specific surface area, pore volume and pore size were calculated from adsorption-absorption isotherms using Brunauer-Joyner-Halenda (BJH) method. FTIR analysis was done on a Nicolet 6700 FT-IR. ¹H-NMR studies were performed on a Bruker DRX-400 spectrometer. Bransonic® 3105E-DTH sonicator form Branson (CT, USA) with a frequency of 40 kHz and an output power of 335 W was used for ultrasonication performed below 30° C. A Brookhaven BI-9000AT Digital Autocorrelator was used for dynamic light scattering measurements. The scattering angle was fixed at 90° for measuring hydrodynamic radius (Rh) which was obtained using a cumulant analysis. The polydispersity index (PDI) of the particles was indicated by the values of μ₂/<Γ>², where μ₂ and Γ are the second cumulant and the first cumulant respectively. To determine the relationship of apparent diffusion coefficient, Dₐₚₚ, of PEG-g-hollow silica vesicles and the square of the wave scattering vector, q², the scattering angel was changed from 50° to 130°. Isocratic reverse-phase high performance liquid chromatography (HPLC) was implemented on the Waters 2695 Separation Module with a reverse phase SymmetryShield® Column (pore size 5 μm, 150×4.6 mm i.d.) and a Waters 2996 PDA detector with Millennium processing software version 3.2. A solvent mixture of acetonitrile-water (50:50, v/v) was used as mobile phase at a flow rate of 1 mL/min at 25° C., and the wavelength of the UV detector was set at 227 nm.

### EXAMPLE 1

### Preparation of Hollow Silica Spheres

Hollow silica can be prepared using various methods reported. One method is using polystyrene latexes as templates.

In a typical process, 3.0 g of PVP was dissolved in 100 mL of HPLC grade water under stirring for 24 h at room temperature. Then 11.0 mL of styrene and 0.26 g of AIBA were added to the solution under stirring at 100 rpm and 70° C. under Argon. After 24 h, 18 mL of polystyrene colloid solution was mixed with 240 mL of ethanol and 12 mL of ammonia solution (NH₄OH) (25 wt %). Then 3.18 mL of TEOS in 5 mL of ethanol was added dropwise, and the mixture was stirred at 50° C. for 24 h. The solid was collected by centrifugation and were calcinated at 550° C. to get hollow silica spheres.

The hollow silica obtained was characterized. FIG. 2B is a TGA curve of the obtained hollow silica, and FIG. 2A is a TEM image thereof. FIG. 7a is a FTIR spectrum of hollow silica. FIG. 6A is the nitrogen adsorption-desorption isotherms of the hollow silica. The average diameter of the pores of the silica shells is 1.19 nm. The surface area and total pore volume are 164 m²/g and 0.23 cm³/g obtained using the BET and BJH methods, respectively.

### EXAMPLE 2

### General Procedure of the Preparation of Hollow Silica Carrying Amino Groups

Typically 2 g of hollow silica was dispersed in 90 ml of xylene. A good dispersion was obtained after ultrasonication for 6 h. After a mixture of 4 ml of 3-aminopropyltriethoxysilanes (APES) (or N-(3-trimethoxysilyl)propyl ethylene diamines (TMSPEA)) and 5 ml of xylene was added dropwise under argon at. 50° C. for 10 min, the solution was kept at 90° C. for 24 h. The solution was precipitated in ether, and the solid was collected by filtration and was purified by washing with ether. After drying, the content of the organic species was evaluated using TGA and the result is listed in FIG. 2b. In comparison with the TGA result of pure hollow silica, the organic species content is around 7%.

### EXAMPLE 3

### Preparation of PEG-Graft-Hollow Silica

### (a) Preparation of Methyl PEG 4-Nitrophenyl Carbonate

10 g of methyl PEG monoether (Mw: 5 000) was dissolved in 100 ml of dichloromethane. Then 1.2 g of 4-nitrophenyl chloroformate and 0.82 ml of triethylamine were added respectively. The solution was stirred at ambient temperature for 7 days. The crude product was purified by recrystallization from chloroform-diethyl ether. NMR (CDCl₃): δ 58.91 (OCH₃), 68.25, 68.54, 70.50, 70.64, 70.90, 71.87 (OCH₂CH₂), 121.75, 125.20, 145.32, 152.37 (aromatic), 155.49 (OCOO).

### (b) Preparation of PEG-Graft-Hollow Silica

PEG-graft-hollow silica is prepared by the approach as summarized in Scheme 1. In a glass flask, 0.8 g of hollow silica containing amino groups (HSilica-NH₂) was mixed with 7.2 g of PEG 4-nitrophenyl carbonate in 100 ml of dimethylsulfone. Then the mixture was heated at 80° C. under argon for 2 days. After cooling down, the solution was precipitated in ether. The solid was collected using centrifugation and washed with fresh DMSO followed by drying under vacuum at 50° C.

### EXAMPLE 4

### Characterization of PEG-Graft-Hollow Silica

FIG. 8 is a ¹H NMR spectrum of PEG-graft-hollow silica recorded in deuterium oxide. The peak at 3.61 ppm is attributed to the protons of PEG grafted onto hollow silica. FIG. 7B is FTIR spectrum of PEG-graft-hollow silica. The characteristic peaks of urethane group at around 1769, 1701 1526, 842 cm⁻¹ can be observed. This indicates that the PEG chains are grafted to the hollow silica via urethane groups as described in FIG. 1. FIG. 2D is the TGA curve of PEG-graft-hollow silica. Via a comparison with FIG. 2B, the content of PEG in PEG-graft-hollow silica is 36%. These results indicate that PEG is grafted onto hollow silica. FIG. 2C shows a TEM image of PEG-graft-hollow silica (cf. FIG. 2A for pure hollow silica). The average diameter of the hollow silica is 190 nm and that of the PEG-graft-hollow silica is 180 nm. The shell of PEG-graft-hollow silica is obviously thicker than that of hollow silica. The average thickness of hollow silica is 16 nm. After grafting of PEG, the average thickness is 37 nm.

### EXAMPLE 5

### Good Dispersion of PEG-g-Hollow Silica in Aqueous Solution

The graft PEG brushes render PEG-graft-hollow silica good dispersion in water. DLLS was applied to characterize the aqueous solution of PEG-graft-hollow silica. FIG. 6 shows that the diffusion coefficient D from DLLS measurements on PEG-graft-hollow silica is almost independent of the scatter vector. This indicates the spherical shape of the PEG-graft-hollow silica and no aggregation of the vesicles in aqueous solution. Rh of PEH-graft-hollow silica is 102.9 nm, and this value is close to that of the individual vesicles obtained using TEM. The silica shells can prevent the effect of dilution on the stability of the vesicles. Also the vesicles have a long shelf-life. No change in Rh of the vesicles can be detected in 6 months.

### EXAMPLE 6

### Loading of Anti-Cancer Drug Paclitaxel

Anti-cancer drug paclitaxel with a very low solubility in aqueous solution was adopted to demonstrate the feasibility of loading active species into the PEG-graft-hollow silica. First a saturated solution of PTX in methanol was prepared and the saturated solubility was determined to be ca. 50 mg/mL. In a typical process, 60 mg of PEG-g-hollow silica was dispersed in 8 ml of saturated solution of paclitaxel in methanol. After ultrasonication for 3 h at a temperature below 30° C., the solution was centrifugated at 8000 rpm for 10 min to collect the solid, and the solid was dried under vacuum at ambient temperature for 30 min. Then the solid was dispersed in 30 ml of DI water under ultrasonication for 4 h at a temperature below 30° C. Then the solution was filtrated through a 5 μm filter and the solution was lyophilized. The process could be repeated once. To measure the loading capacity, 3 mg of the obtained solid was dispersed in 1 ml of dichloromethane in a vial under ultrasonication for 3 h at a temperature below 30° C. Then the vial was kept open to let dichloromethane evaporate. Subsequently the solid was dissolved in 2 ml of a 50:50 (v/v) mixture of water and acetonitril under ultrasonication for 30 minutes for HPLC measurement. The retention time of the peak of PTX is ca. 8.2 minute. Concentration of PTX was obtained from a calibration curve, which is linear over the concentration of PTX from 0.01 to 0.62 mg/mL with a correlation coefficient of R²=0.993.

The silica shell is nanoporous with an average pore diameter of 1.9 nm. Therefore the saturated solution of paclitaxel in methanol can fill the pores and the hollow cores. The capillary forces of the nanosized pores should make the filling feasible. After removing methanol, paclitaxel is loaded into PEG-graft-hollow silica. The loading processes can be repeated. After one time of loading, the content of paclitaxel in the product is 3.2%. After the second loading, the content is 4.5%. The vesicles loaded with paclitaxel still have good dispersion in aqueous solution without aggregation.

In order to investigate whether the aggregates of free PTX in the aqueous solution can be removed by the filtration through membranes with pores of 5 μm diameter, 10 mg of PTX solid from drying the saturated solution of PTX in methanol was dispersed in 10 mL of DI water under ultrasonication for 6 h. The solution was filtrated through membranes with pores of 5 μm diameter. The filtrate was lyophilized, and the solid obtained was determined to be negligible using HPLC. This indicates that the aggregates of free PTX in aqueous solution could be removed by the filtration.

### EXAMPLE 7

### Release Profile of Anti-Cancer Drug Paclitaxel

In a typical process, 0.7 mg of paclitaxel loaded PEG-graft-hollow silica was dispersed in 3 ml of 1×PBS buffer solution under ultrasonication for 2 h at a temperature below 30° C. The solution was transferred into a dialysis tube with a molecular weight cutting of 10 000. The dialysis tube was placed into a conical flask containing 40 ml of 1×PBS buffer solution. The conical flask was kept in a water bath at 37° C. and was stirred at 100 rpm. The outside buffer solution was exchanged with a fresh buffer solution at a predetermined time interval to maintain a sink condition. The released paclitaxel was extracted from the drawn buffer solution using 4 ml of dichloromethane. After shaking and setting down, the dichloromethane solution was collected. After chloromethane was evaporated at room temperature, the solid reconstituted in 2 mL of acetonitrile/water (50:50; v/v) for HPLC measurement to determine PTX amounts.

To measure the release profile of free PTX, 2 mg of PTX was firstly dissolved in 4 mL methanol to get a solution of a concentration of 0.5 mg/mL. After ultrasonication for 40 min, 0.4 mL of the solution was taken out and was dried by evaporating methanol. The solid was dissolved in 3 mL of 1×PBS solution (pH 7.4), and the release profile was monitored.

FIG. 3 (curve b) shows the release profile of the loaded paclitaxel from PEG-graft-hollow silica. A controlled release of paclitaxel is observed. Less than half of the loaded paclitaxel was released after 330 h. In contrast to the control experiment of paclitaxel powder as shown in FIG. 3 (curve a), the release rate of paclitaxel from PEG-g-hollow silica was significantly improved. The slow release rate of paclitaxel powder resulted from the low solubility of paclitaxel in water. The improved paclitaxel release rate should be contributed to the nanosized form of paclitaxel encapsulated in PEG-g-hollow silica due to the increased surface area of the nanosized paclitaxel hold in the nanosized hollow silica. These results also confirm that paclitaxel can be encapsulated in PEG-g-hollow silica.

In addition, the longer fluorescence lifetime also renders the obtained nanoparticles a new type of nanoprobes in time-resolved fluoroimmunoassays with increased S/N ratio (i.e. the assay sensitivity). Overall, the resulting colloidal rare-earth-incorporated metal oxide nanoparticles are also identified here as a promising alternative of prevailing luminescent probes like organic dyes and semiconductor quantum dots as bioprobes/nanotags/nanolabels for imaging, sensing, and diagnostics besides lighting applications.

### EXAMPLE 8

### In Vitro Cytotoxicity Evaluation of Samples

Cytotoxicity of pure PTX, PEG-g-hollow silica vesicles, and PTX loaded PEG-g-hollow silica vesicles was evaluated in MCF-7 cell lines and U-87 MG cell lines. Viability of the cells was assessed by the standard thiazolyl blue [3-(4,5-dimethyliazolyl-2)-2,5-diphenyl tetrazolium bromide] (MTT) assay. This colorimetric assay allows determination of the number of viable cells through the metabolic activity of the cells.

The cancer cells were seeded in 96-well plates with a seeding density 10,000 cells/well and were cultured in DMEM supplemented with 10% FBS and 1% penicillin/streptomycin solution in an incubator at 37° C., 5% CO₂, and 95% relative humidity. The cells were allowed to adhere to the well bottom upon overnight incubation. Then the medium was replaced with the sample solutions of different concentrations. Meanwhile, wells containing only cell culture medium were prepared as untreated controls. At the predetermined time, the medium containing samples was aspirated and the wells were washed with 1×PBS solution for two times to removed non-internalized sample. Then 100 μL of DMEM and 10 μL of MTT solution (5 mg/mL in 1×PBS solution) were added to the wells. After incubation for 4 h at 37° C., the solution was removed and the formazan precipitate was dissolved in 100 μL of dimethyl sulfoxide (DMSO). The absorbance intensity of the solution was then quantified spectrophotometrically using a microplate reader (TECAN SpectraFluor Plus) at 570 nm. Cell viability was expressed by the following equation:

Cell viability (%)=Absₛₐₘₚₗₑ/Abs_{control}×100%

where Absₛₐₘₚₗₑ was the absorbance for the cells treated with the samples, while Abs_{control} was the absorbance for the untreated control cells. All the tests were performed in triplicate.

### EXAMPLE 9

### Incubation of Mammalian Cells with FITC Labeled PEG-g-Hollow Silica Vesicles

MCF-7 breast cancer cells and U-87 MG brain cancer cells are seeded on the chambers (Lab-Tek® Chambered Coverglass System, 8 chambers) at a density of 1×10⁴ cells/well in DMEM supplemented with 10% FBS and 1% antibiotics (penicillin/streptomycin). After incubation overnight at 37° C. in a humidified atmosphere with 5% CO₂, the medium was aspirated off. Then, 250 μL of 200 μg/mL FITC labeled PEG-g-hollow silica vesicles in DMEM without serum was added. After incubation for 18 h, the medium was removed and the cells were gently rinsed with 1×PBS solution. Then the cells were fixed with 250 μL of 0.3% glutaraldehyde/PBS solution for 10 min. After the fixative solution was removed, the cellular nucleus was then stained by DAPI containing ProLong® Gold antifade reagent in the dark for 15 min. Laser confocal fluorescence micrographs were obtained on a Carl Zeiss LSM 5 DUO, Inverted Stage. For DAPI imaging, the emission was observed at 421 nm with an excitation at 401 nm; and for FITC imaging, the emission was observed at 517 nm with an excitation 495 nm.

### EXAMPLE 10

### The Procedure to Calculate the Theoretical PTX % Loaded in PEG-g-Hollow Silica Vesicles

From BET, the volume of pores in the silica shell is Vₚₒᵣₑ is 0.24 mL/g.

From TEM images, the average radius of the hollow silica, R₁, and the interior, R₂, are 95 nm and 88 nm, respectively. So for a single hollow silica sphere,

$$\begin{matrix}{SV}_{interior} = \frac{4}{3} π {R}_{2}^{3} \\ = \frac{4}{3} π \times {( 88 \times {10}^{- 7} )}^{3} \\ = 2.85 \times {10}^{- 15} ( mL )\end{matrix}$$

$$\begin{matrix}{SV}_{silica} = \frac{4}{3} π ( {R}_{1}^{3} - {R}_{2}^{3} ) \\ = \frac{4}{3} π ( {95}^{3} - {88}^{3} ) \times {10}^{- 21} \\ = 7.36 \times {10}^{- 16} ( mL )\end{matrix}$$

$$\begin{matrix}{SW}_{silica} = {SV}_{silica} \times {d}_{silica} \\ = 7.36 \times {10}^{- 16} \times 2.2 \\ = 1.62 \times {10}^{- 15} ( g )\end{matrix}$$

where SVᵢₙₜₑᵣᵢₒᵣ and SV_{silica} are the volume of the interior and the silica shell of a single hollow silica sphere, and SW_{silica} is the weight of a single hollow silica sphere.

Hence, Vᵢₙₜₑᵣᵢₒᵣ and Vₚₒᵣₑₛ, the volume of the interior and pores in hollow silica sphere of W_{silica} gram:

$${V}_{interior} = \frac{{SV}_{interior}}{{SW}_{silica}} \times {W}_{silica} = 1.76 {W}_{silica} ( mL )$$

$${V}_{pores} = 0.24 {W}_{silica} ( mL )$$

$$From TGA , \frac{{W}_{silica}}{{W}_{silica} + {W}_{PEG}} = 46 \%$$

where W_{silica} and W_{PEG} are the weight of the silica shell and the PEG brushes, respectively.

Hence, the theoretical PTX % loaded in PEG-g-hollow silica vesicles,

$$\begin{matrix}PTX \% = \frac{{V}_{pore} + {V}_{interior}}{{W}_{silica} + {W}_{PEG}} \times S \times 100 \% \\ = \frac{1.76 {W}_{silica} + 0.24 {W}_{silica}}{{W}_{silica} + {W}_{PEG}} \times S \times 100 \% \\ = ( 0.243 + 1.76 ) \times 0.46 \times 50 \times {10}^{- 3} \times 100 \% \\ = 4.6 \%\end{matrix}$$

where S is the solubility of PTX in the loading medium.

In conclusion, in the above illustrative examples PEG-g-hollow silica vesicles were obtained by grafting PEG to hollow silica spheres. PEG-g-hollow silica vesicles have a good dispersity in aqueous solution rendered by PEG stealth layers and a good stability due to the silica layers. The porous silica layers make it feasible to load water-insoluble drug PTX, and PTX can be released faster from nanosized PEG-g-hollow silica vesicles than free PTX. While PEG-g-hollow silica vesicles show a very low in vitro cytotoxicity in cells, PTX loaded PEG-g-hollow silica vesicles show a potent capacity to kill cancer cells probably attributed to the capability of PEG-g-hollow silica vesicles to internalize cells and the fast release of PTX. Moreover, polymer functionalized hollow silica vesicles promising for many applications can be obtained through tuning the chemistry and structures of the polymer layers and silica layers.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognised that various modifications are possible within the scope of the invention claimed. Additional objects, advantages, and features of this invention will become apparent to those skilled in the art upon examination of the foregoing examples and the appended claims. Thus, it should be understood that although the present invention is specifically disclosed by exemplary embodiments and optional features, modification and variation of the inventions embodied therein herein disclosed may be resorted to by those skilled in the art, and that such modifications and variations are considered to be within the scope of this invention. In addition, where features or aspects of the invention are described in terms of Markush groups, those skilled in the art will recognise that the invention is also thereby described in terms of any individual member or subgroup of members of the Markush group.

1. A hollow silica micro- or nanoparticle with a polymer immobilized thereon, wherein the polymer is covalently linked to the silica particle via one or more bridges that have an urethane group.

2. The silica particle of claim 1, wherein the surface of the silica particle has a polymer content in the range from about 10% to about 90% (w/w).

3. The silica particle of claim 1, wherein the surface of the silica particle has a polymer content of at least about 35% (w/w).

4. The silica particle of claim 1, wherein the polymer is a polar polymer.

5. The silica particle of claim 4, wherein the polymer is one of poly(ethylene glycol), polyglycolic acid (PGA), polylactic acid (PLA), polycaprolactone, or a polyhydroxyalkanoate.

6. The silica particle of claim 1, wherein the bridge via which the polymer is coupled to the surface of the particle is one of an aliphatic, an alicyclic, an aromatic or an arylaliphatic bridge and wherein the link of the polymer to the silica particle is of Formula (1) 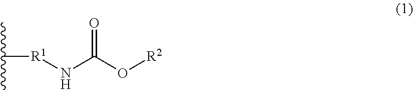
wherein R² is the polymer and R¹ is one of an aliphatic, an alicyclic, an aromatic or an arylaliphatic bridge, having a main chain of about 1 to about 30 carbon atoms and 0 to about 10 heteroatoms selected from the group consisting of N, O, S, Se and Si.

7. The silica particle of claim 1, wherein the silica particle has a maximal width of about 1 nm to about 1 μm.

8. The silica particle of claim 1, wherein the particle is microporous or mesoporous.

9. The silica particle of claim 1, wherein the hollow particle has an inner void that comprises a pharmaceutically active compound.

10. A pharmaceutical composition comprising a plurality of hollow silica particles, wherein each of the plurality of hollow silica particles comprises a hollow silica micro- or nanoparticle with a polymer immobilized thereon, wherein the polymer is covalently linked to the silica particle via one or more bridges that have an urethane group, wherein the hollow silica particle has an inner void that comprises a pharmaceutically active compound.

11. A method of covalently coupling a polymer to a silica surface, the method comprising:
providing a silica surface that carries amino functional groups,
providing a polymer with a carbonate group of the general Formula (2), 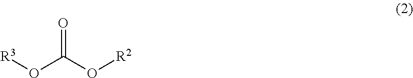
wherein R² is the polymer and R³ is one of an aliphatic, an alicyclic, an aromatic, an arylaliphatic group or a silyl group, having a main chain of about 1 to about 20 carbon atoms and 0 to about 6 heteroatoms selected from the group consisting of N, O, S, Se and Si,
contacting the polymer and the silica surface, and
allowing the carbonate group of the polymer and an amino functional group on the silica surface to undergo a coupling reaction, thereby covalently coupling the polymer to the silica surface.

12. The method of claim 11, wherein the silica surface is the surface of a micro- or nanoparticle.

13. The method of claim 11, wherein the micro- or nanoparticle is a hollow particle.

14. The method of claim 11, wherein R³ is one of a nitrobenzyl-, a trifluoromethyl, or a succinimde group.

15. The method of claim 11, wherein the polymer is a polar polymer.

16. The method of claim 11, wherein providing a silica surface that carries amino functional groups comprises:
providing a silica surface,
providing a compound of the general Formula (3), 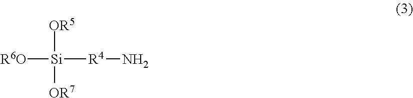
wherein R⁴ is one of an aliphatic, an alicyclic, an aromatic or an arylaliphatic bridge, having a main chain of about 1 to about 10 carbon atoms and 0 to about 5 heteroatoms selected from the group consisting of N, O, S, Se and Si, and R⁵, R⁶ and R⁷ are independent from one another, an aliphatic group with a main chain of about 1 to about 10 carbon atoms and 0 to about 3 heteroatoms selected from the group N, O, S, Se and Si,
contacting the compound of the general Formula (3) and the silica surface, and
allowing at least one of the groups OR⁵, OR⁶ and OR⁷ of the compound of the general Formula (3) to undergo a coupling reaction with the silica surface, thereby forming a covalent bond with the same.
